(19) United States
(12) Patent Application Publication
(10) Pub. No.: US 2023/0090672 A1
(43) Pub. Date: Mar. 23, 2023

(54) RAPID AND SAMPLE-SPECIFIC DETECTION OF VIRAL PATHOGEN FOR POOLED TESTING IN LARGE-POPULATION SCREENING

(71) Applicant: THE HONG KONG UNIVERSITY OF SCIENCE AND TECHNOLOGY, Hong Kong (CN)

(72) Inventors: I-Ming HSING, Hong Kong (CN); Xinyu ZHUANG, Hong Kong (CN); Xiao LU, Hong Kong (CN); Henson Lim LEE YU, Hong Kong (CN)

(21) Appl. No.: 17/807,822

(22) Filed: Jun. 20, 2022

Related U.S. Application Data
(60) Provisional application No. 63/212,719, filed on Jun. 20, 2021.

(51) Int. Cl. C12Q 1/6844; C12Q 1/686; C12Q 1/6818

(52) U.S. Cl. CPC C12Q 1/6844; C12Q 1/686; C12Q 1/6818

(57) ABSTRACT

The subject invention pertains to methods for the analysis of pooled samples without the need of retesting through the use of oligonucleotide hybridization and target-specific amplification reactions. Specifically, a series of identifier oligonucleotides with different sequence compositions, each corresponding to a distinct sample, are combined into the target template of interest through nucleic acid synthesis. The aforementioned products are pooled together, and the pooled samples are amplified and detected using the probe-based hybridization assay or a size separation module to identify if any of the pool of samples test positive, as well as simultaneously identifying which sample is positive for the targeted sequence.

### CROSS-REFERENCE TO RELATED APPLICATION

This application claims the benefit of U.S. Provisional Application Ser. No. 63/212,719, filed Jun. 20, 2021, which is hereby incorporated by reference in its entirety including any tables, figures, or drawings.

### BACKGROUND OF THE INVENTION

Rapid host-to-host transmission coupled with the ease of international travel have been the root cause of epidemics such as H1N1, Zika, and SARS, and most recently, the COVID-19 pandemic¹. Given the magnitude of crisis, even developed countries are often unable to provide timely vaccination and treatments to a sufficient scale in order to contain an infectious disease². Furthermore, the development of a reliable vaccine usually takes years or months³. Thus, the world still relies on classic epidemic control measures, such as contact tracing and physical isolation, to mitigate the spread of the disease⁴, which are highly dependent on the accuracy and rate of diagnosing suspected patients⁵. Currently, the gold standard for pathogen detection is still nucleic acid-based polymerase chain reaction (PCR)⁶-7. However, factors such as limited testing capacity, high cost, and long assay time for a single PCR test often compromise its effectiveness to curb the community-wide transmission, particularly in resource-poor regions⁸.

Alternatively, pooled testing, in which multiple individual samples are grouped together and tested, is an attractive way to scale up the diagnostic capacity and save the operation time and cost. This strategy was first proposed by Dorfman, who suggested that samples can be pooled together and tested at the same time to reduce the total number of tests performed⁹. If the pooled sample tests negative, it implies that the individual samples in the pool are negative; whereas, if the pooled sample is positive, then each sample must be tested individuallyl¹⁰. Dorfman testing has been applied in HIV screening in blood donors¹¹, detection of Salmonella in feces¹², and influenza viruses¹³. Even for the recent COVID-19 outbreak, many countries and regions have employed pooled testing to address the overwhelming need for rapid and mass community testing^{14,15}.

Although it has been reported that pooling samples into groups of 4 to 10 reduces the number of tests by 50%-60%¹⁶, there are certain limitations. First, sensitivity is reduced because of the dilution from negative samples¹⁷; and second, selectivity is compromised due to a higher probability of cross-contamination¹⁸. Moreover, optimizing the number of samples to pool together requires a prior estimation of the prevalence of the disease¹⁹; because, if the size of the pool is too large and/or the prevalence of the disease is too high, it will not reduce the overall number of tests, as many pooled samples require retesting. Many studies have reported improvements in the sample pooling performance by using mathematical and statistical algorithms, for example, to predict the optimal pool size⁸ on the basis of carrier rate²⁰; however, this still does not eliminate the need for the retest step. Additionally, massively parallel diagnostic assays that combine multiplex barcoding and next-generation sequencing (NGS) technique^{21,22} have been applied to achieve sample-specific detection of viral pathogens. Hossain and his colleagues developed a barcoded sequencing approach for simultaneous testing of 19,200 patient samples for COVID-19 screening²³. While NGS is able to achieve high throughput, expensive NGS consumables and the associated requirement of bioinformatics tools along with manpower limits its broad deployment and applicability in large-scale settings.

Therefore, there is a need for more efficient pooled testing platform that minimizes, if not eliminates, the need for secondary individual re-testing. Furthermore, this method must be rapid and involve less-sophisticated diagnostic platforms, thereby utilizing commonly employed instruments.

### BRIEF SUMMARY OF THE INVENTION

The subject invention pertains ID-Primer assisted sample-specific pooled testing strategy (Uni-Pool) in which target genetic sequences of, for example, a viral pathogen in a crude sample, can be extracted and amplified with tagging of sample-specific identifiers. The subject methods offer a sample-specific readout by incorporating a sample-specific identifier strand of different sequences (i.e., with distinguishable Gibbs free energy and/or melting temperature) to the 5′ end of a primer complementary to the target nucleotide sequence, such as, for example, viral RNA. The subject methods can utilize rationally designed labelled oligonucleotide probes, such as, for example, fluorescence- and quencher-tagged and fluorescence channels, the workflow of which is compatible to reverse-transcription real time PCR (RT-qPCR) for viral RNA pathogens. Each sample outcome (positive or negative) in one pool can be obtained by high-resolution melting curve analysis (HRMCA)^{24,25} after qPCR.

The subject methods can eliminate the need for re-testing and allowing the test results, positive or negative, for all samples in the pool to be revealed by multiplex melting curve analysis after real-time PCR. The subject methods can further significantly reduce total assay time for largescale screening without compromising specificity and detection sensitivity caused by the sample dilution of pooling.

In certain embodiments, at least 2-50 samples can be differentiated, positive and negative, in one pool with negligible cross-reactivity among positive and negative samples.

### BRIEF DESCRIPTION OF THE DRAWINGS

FIGS. 1A-1C. FIG. 1A. The workflow of unique ID-Primers assisted sample-specific pooled testing (Uni-Pool). Each of the extracted samples is added with a sample-specific ID-Primer and then collected in one tube for pooling, followed by real-time PCR amplification and multiplexed melting curve analysis. FIG. 1B. A detailed look of reverse transcription (RT). The first step involves adding the ID-Primer to each sample by RT. At this stage, the samples with viral RNA will generate a cDNA-RNA duplex. FIG. 1C. Analysis of Uni-Pool through closed tube 2D multiplex PCR. Five sets of melting curve detection probes with different colors (FAM and HEX) are used to identify the matching templates amplified with ID-Primers. In the pool, the cDNA-RNA templates will generate abundant complementary single strand amplicons through asymmetric PCR with abundant forward primer. Next, the matching fluorophore and quencher probe sets hybridize to the homologous amplicons at 45° C. By increasing the reaction temperature, the first derivative of the fluorescent intensity vs. temperature (−dRFU/dT) is an indication of positive (with peak) or negative (without peak) of the screening outcome.

FIGS. 2A-2K. The melting curve plots of one pool with three ID-Samples in single FAM channel. The concentration of synthetic RNA template used here is 103 copies/μL, which is the mean viral load of SARS-CoV-2 in saliva specimens. RFU refers to relative fluorescence unit. FIGS. 2A-2F. Comparison of melt peak signal from the melting curve analysis using FRET probes (FIGS. 2A-2C) and the self-quenched TaqMan probe (FIGS. 2D-2F). The red lines in a, b, c and d are results of the pool with three positive samples and the black lines refer to the results of no template control (NTC). RFU-T (FIGS. 2B and 2E) is the record of fluorescence signal changes with temperature and −dRFU/dT (FIGS. 2C and 2F) is the change rate of fluorescence signal. For the self-quenched TaqMan probe, when it hybridizes with the target sequence, the fluorescence signal is emitted because the fluorophore is far away from the quencher. After heating, once the temperature reaches the melting temperature (Tₘ) of probes, it can be released from the probe-target duplex, thus the fluorescence signal will decrease sharply because of the free state of probe structure. For the FRET probes, the fluorescence signal change is opposite to that of self-quenched TaqMan probe. FIGS. 2G-2K. The melting curves of the three pooled targets detected by the FAM channel, including sample 1, sample 2 and sample 3, are displayed. All the red lines correspond to the target signals in FAM channel and the black lines refer to the PCR NTC. Tₘ of the melting valleys corresponding to sample 1, sample 2 and sample 3 are 48° C., 60° C., 70° C., respectively. The ‘positive’ and ‘+’ mean the sample has RNA and ‘negative’ and ‘−’ mean the sample does not have RNA.

FIGS. 3A-3G. Melting curve analysis of a Uni-Pool containing five samples at two fluorescent channels, FAM and HEX. FIGS. 3A-3B. Two exemplary pools with different number of positive samples (Pool 1: five positive samples, Pool 2: 2nd and 4th samples are positive, the rest are negative). Red lines refer to the samples in FAM, the blue lines are in HEX, and the black lines are no template control (NTC). FIGS. 3C-3F. Sensitivity test of Uni-Pool of five specimens. FIGS. 3C-3D. Melting curve results of the pool with one positive and four negative samples, and the positive one having different concentrations. FIGS. 3E-3F. In the scenario of two positive and three negative samples with these positive samples having Tₘ at 50° C. and 60° C. or 60° C. and 70° C., respectively. In these two positive samples, one is fixed at a concentration of 10⁶ copies/μL, the other is at different template concentrations ranging from 50 copies/μL to 10⁶ copies/μL. FIG. 3G. Cross-reactivity of the heatmap shows the intensity of melting peak height two different concentrations (105 and 103 copies/μL) of synthetic SARS-CoV-2 RNA samples and Influenza H1N1 RNA samples using the primer sets (ID-Primers and PCR primers) for SARS-CoV-2.

FIGS. 4A-4AC. Comparison between detection of 40 simulated saliva samples using Uni-Pool assay and conventional group test. FIG. 4A. Schematics of detection of 40 simulated saliva samples using our Uni-Pool and the conventional group test. The 40 simulated saliva samples with a prevalence of 15% were randomly divided into 8 pools containing five individual samples. For the Uni-Pool assay, the extracted samples first were reverse transcribed in which an ID-Primer acted as a primer for the reverse transcriptase and then pooled for one-pot multiplex melting curve analysis. For the conventional Dorfman-based group test, the extracted samples will be pooled together and analyzed by the one-step RT-qPCR, and an additional step is the retest of the individual samples in the pools with positive feedbacks. FIGS. 4B-4G. Detection results of 40 saliva samples using 2D Uni-Pool assay and conventional group test. In Pool 1: the sample 1 has 10 copies/μL pseudovirus and others are negative. In pool 8: the sample 36 has 50 copies/μL pseudovirus, the sample 40 has 103 copies/μL pseudovirus and others are negative. For the conventional group test, the Cq value less than 40 indicates a positive result of SARS-CoV-2. All reactions were performed in triplicates and detail results are summarized in FIGS. 4H-4AC.

FIG. 5. The structure of the ID-Primer. The rationally designed oligonucleotide sequence consists of three core parts: the target-specific region complementary to the RNA target of interest, the ID region that provides a unique identifier sequence and can be differentiated by its melting temperature (by altering the sequence and the length), and the amplifier sequence for the subsequent PCR primer binding region. The ID region is designed to be orthogonal to each other to eliminate primer dimerization, and the amplifier sequence is also designed to be different to eliminate the bias from the varying target concentration in different samples.

FIG. 6. The principle of adding ID-Primers to each and sample pooling. First, a unique ‘ID-Primer’ is added to each sample after RNA extraction. If the sample contains the viral RNA, as illustrated in samples 1 and 3, the ID Primer will act as the reverse transcription primer, thus generating a cDNA-RNA duplex upon the addition of reverse transcriptase without Ribonuclease H (RNase H) activity. On the contrary, the primers remain as single-stranded DNA without the RNA target (as illustrated in sample 2). Then, exonuclease I is added to each sample. Since it can only degrade single stranded DNA (from 3′ to 5′ end), it will selectively digest all unused ID-Primers and only the cDNA-RNA duplexes remain intact. Therefore, each sample that is positive for the viral RNA target will be labeled with a special tag and can then be differentiated after being pooled in the next step.

FIG. 7. The detection scheme of pooled samples using probe-based melting curve analysis. The strategy is designed for the sample pooling using the primers with different Tₘ value in the ID region. First, the samples are amplified via asymmetric PCR to generate single-stranded amplicons from the cDNA targets in the pool. The melting curve probe set comprises a probe with the fluorophore (complementary to the ID region) and a probe with the quencher (complementary to the cDNA region adjacent to the F probe-binding region). The Q probe is long enough to make sure that its Tₘ value is around 80° C. During the melting curve analysis, the F and Q probes will hybridize to their corresponding sites at around 45° C., and at this point, the fluorescence signal is quenched. When the temperature reaches the Tₘ value of the F probe, the F probe will be released and would yield a sharp increase in fluorescence signal. By designing different fluorophores and Tₘ values of the F-probe, the melting curve peak can be correlated to the presence of the specific tagged cDNA target.

FIG. 8. The detection scheme of pooled samples using fragment length identification. The strategy is designed for the sample pooling using the primers with different lengths of the ID region. After sample pooling, the first step involves the PCR or isothermal amplification of the pooled samples to produce the double strand amplicons with unique lengths that can be traced to the length of the ID-Primers initially added. Thus, in this step, the initial RNA/DNA duplex will be converted to a double-stranded DNA molecule. This product is then detected using the fragment analyzer. By comparing the bands in a sample to the DNA ladder, the positive samples can be inferred by the approximate size of products which amplify from RNA/DNA duplexes. If no band is detected, then all individual samples are declared negative, but if the band is present in the expected position, the length of the product will reveal which sample is positive.

FIG. 9. The detection scheme of pooled samples using real-time PCR. The strategy is designed for the sample pooling using the tag primers with different base combinations in the ID regions around 20-30 bp. During the PCR amplification process, the TaqMan probes labeled with different types of fluorophores will hybridize with the expected targets and can be hydrolyzed by the exonuclease activity of the polymerase to give out the real time fluorescent signals. If the viral RNA target exists, there will exhibit a sign of exponential growth. According to the cycle threshold value, this assay can achieve quantitative and qualitative analysis of grouped samples.

FIG. 10. The detection scheme of pooled samples using electrochemical detection. The strategy involves adding ID-Primers around 20-50 bp with different base compositions. First, the grouped cDNA samples will be mixed with amplification primers and homologous probes that labeled with electroactive reporters with non-overlapping redox potentials, i.e., ferrocene, methylene blue, anthraquinone. Because the DNA polymerase has exonuclease property, the electrochemical probes that hybridized with the correct targets will be hydrolyzed during the extension step. Thus, the electroactive reporters with short mononucleotides can be released. After amplification, the end point electrochemical test will be carried out. As a result, the virus-contained sample can be differentiated from the virus-free sample by the high current peak at certain potential.

FIG. 11. The principle of hybridization-based adding of ID-Primers and sample pooling. In this assay, the adding of ID-Primers is achieved through the specific hydrogen bonding patterns of nucleobases and the sample pooling step is performed before the reverse transcription reaction. The target specific ID-Primers (FIG. 5) are added to the extracted RNA templates. The secondary structures of single strand RNA templates are loosed by heating at 65° C. and then the ID-Primers are annealed to the complementary RNA strands at lower temperature. After annealing, the exonuclease I is used to digest the unhybridized ID-Primers to prevent cross-reactivity in the grouped samples. Then, all ID-Samples will be mixed with equal volume and then detected using the abovementioned detection schemes (FIGS. 7-10).

FIG. 12. The principle of sample pooling detection using the Magnetic Bead (MB) system. This step combines sample preparation and adding ID-Primers into one step which involves the extraction-free sample preparation method for raw RNA samples. The lysis buffer is added to the collected samples from individuals. After incubation, nucleic acids, proteins, and other substances are released. The biotin labeled capture strand will first be attached to the streptavidin coated magnetic bead (MB) through the streptavidin-biotin interaction. Then the streptavidin coated magnetic beads linked with capture strands and ID-Primers (FIG. 5) are introduced to the mixtures, and the RNA targets will be specifically hybridized with the capture strand. At the same time, the ID-Primers will also bind to different regions of RNA targets. After that, the unbound primers and impurities are washed away that doesn't rely on enzyme digestion, and only the DNA-RNA duplexes are preserved. Next, all the samples will be pooled together and then go through the reverse transcription and detection steps (FIGS. 7-10). Furthermore, the MB can also be separated from the system by the displacement activity of reverse transcriptase. After multiplexed reverse transcription, the magnetic field can be used to separate the tagged cDNA targets and MB with RNA capture strand.

FIG. 13. The working principle of the universal melting curve detection probes. The F probe is designed to be fully complementary to the ID region of the sample 1. When this F probe hybridizes with the sample 2, it will form a 2 nts dangle end and a 4 nts dangle end with sample 3. This 2 nts dangle can lead to 5° C. decreases in the Tₘ value during the melting curve analysis.

FIG. 14. The gel electrophoresis results of detection of pooled samples using fragment length identification. Three ID-Primers with different lengths were added to three individual samples through reverse transcription. If three samples have viral RNA, they will generate three amplicons with 132 bp, 120 bp, 99 bp respectively. The pools with different positive rates can all be differentiated by the location of bands in gel electrophoresis.

FIG. 15. The qPCR results of individual barcoded samples after reverse transcription barcoding. The red dots refer to the samples with viral RNA and the blue dots are for the samples without RNA. When the Cq value is below 40, the sample will be regarded as the ‘positive’ case. When the Cq value is above 40, the sample will be considered as the ‘negative’ case.

### BRIEF DESCRIPTION OF THE SEQUENCES

SEQ ID NO: 1: Synthetic RNA(EURO-019) sequence

SEQ ID NO: 2: PCR universal Forward primer to amplify templates with ID-Primers

SEQ ID NO: 3: Multiplex ID-Primer 1 for the first sample in one pool

SEQ ID NO: 4: Multiplex ID-Primer 2 for the second sample in one pool

SEQ ID NO: 5: Multiplex ID-Primer 3 for the third sample in one pool

SEQ ID NO: 6: Multiplex ID-Primer 4 for the fourth sample in one pool

SEQ ID NO: 7: Multiplex ID-Primer 5 for the fifth sample in one pool

SEQ ID NO: 8: Reverse Primer 1 to amplify samples with ID-Primer 1

SEQ ID NO: 9: Reverse Primer 2 to amplify samples with ID-Primer 2

SEQ ID NO: 10: Reverse Primer 3 to amplify samples with ID-Primer 3

SEQ ID NO: 11: Reverse Primer 4 to amplify samples with ID-Primer 4

SEQ ID NO: 12: Reverse Primer 5 to amplify samples with ID-Primer 5

SEQ ID NO: 13: Fluorophore probe 1 to detect amplicons with ID-Primer 1

SEQ ID NO: 14: Fluorophore probe 2 to detect amplicons with ID-Primer 2

SEQ ID NO: 15: Fluorophore probe 3 to detect amplicons with ID-Primer 3

SEQ ID NO: 16: Fluorophore probe 4 to detect amplicons with ID-Primer 4

SEQ ID NO: 17: Fluorophore probe 5 to detect amplicons with ID-Primer 5

SEQ ID NO: 18: Quencher probe to detect amplicons

SEQ ID NO: 19: SARS-CoV-2 N Gene Pseudovirus sequence

SEQ ID NO: 20: N gene forward primer to amplify SARS-CoV-2 N Gene

SEQ ID NO: 21: N gene reverse primer to amplify SARS-CoV-2 N Gene

SEQ ID NO: 22: N gene TaqMan probe to detect SARS-CoV-2 N Gene

### DETAILED DISCLOSURE OF THE INVENTION

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the terms “about” is used, these compositions contain the stated amount of the ingredient with a variation (error range) of 0-10% around the value (X±10%). In other contexts the term “about” is provides a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, an “isolated” or “purified” compound is substantially free of other compounds. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

The terms “label,” “detectable label, “detectable moiety,” and like terms refer to a composition detectable by spectroscopic, photochemical, biochemical, immunochemical, chemical, or other physical means. For example, useful labels include fluorescent dyes (fluorophores), luminescent agents, electron-dense reagents, enzymes (e.g., as commonly used in an ELISA), biotin, enzymes acting on a substrate (e.g., horseradish peroxidase), digoxigenin, ³²P and other isotopes, haptens, and proteins which can be made detectable, e.g., by incorporating a radiolabel into the peptide or used to detect antibodies specifically reactive with the peptide. The term includes combinations of single labeling agents, e.g., a combination of fluorophores that provides a unique detectable signature, e.g., at a particular wavelength or combination of wavelengths. In the context of detecting nucleic acids (e.g., target sequences), the probes can, typically, be labeled with radioisotopes, fluorescent labels (fluorophores), or luminescent agents. The term includes combinations of single labeling agents, e.g., a combination of fluorophores. A unique identifier is a sequence of about 5 to about 30 nucleotides, about 10 to about 25 nucleotides, or about 15 to about 20 nucleotides that are used to distinguish between different samples during sequence analysis.

As used herein, the term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

As used herein, the term “isolated nucleic acid” molecule refers to a nucleic acid molecule that is separated from other nucleic acid molecules that are usually associated with the isolated nucleic acid molecule. Thus, an “isolated nucleic acid molecule” includes, without limitation, a nucleic acid molecule that is free of nucleotide sequences that naturally flank one or both ends of the nucleic acid in the genome of the organism from which the isolated nucleic acid is derived (e.g., a cDNA or genomic DNA fragment produced by PCR or restriction endonuclease digestion). In addition, an isolated nucleic acid molecule can include an engineered nucleic acid molecule such as a recombinant or a synthetic nucleic acid molecule. A nucleic acid molecule existing among hundreds to millions of other nucleic acid molecules within, for example, a nucleic acid library (e.g., a cDNA or genomic library) or a gel (e.g., agarose, or polyacrylamide) containing restriction-digested genomic DNA, is not an “isolated nucleic acid”.

As used herein and in the claims, a “sample” refers to a sample of cell, tissue or fluid, including but not limited to, for example, skin, plasma, serum, spinal fluid, lymph fluid, synovial fluid, urine, tears, blood cells, organs, tumors, environmental sources, including waterways, sewage, soil, or air, samples of in vitro cell culture constituents (including but not limited to conditioned medium resulting from the growth of cells in cell culture medium, recombinant cells and cell components), or any other source derived from an organism or containing an organism.

The term “organism” as used herein includes viruses, bacteria, fungi, plants and animals. Additional examples of organisms are known to a person of ordinary skill in the art and such embodiments are within the purview of the materials and methods disclosed herein. The assays described herein can be useful in analyzing any genetic material obtained from any organism.

“Subject” refers to an animal, such as a mammal, for example a human. The methods described herein can be useful in both humans and non-human animals. In some embodiments, the subject is a mammal (such as an animal model of disease), and in some embodiments, the subject is a human. The terms “subject” and “patient” can be used interchangeably. The animal may be for example, humans, pigs, horses, goats, cats, mice, rats, dogs, apes, fish, chimpanzees, orangutans, guinea pigs, hamsters, cows, sheep, birds, chickens, as well as any other vertebrate or invertebrate.

The term “genome”, “genomic”, “genetic material” or other grammatical variation thereof as used herein refers to genetic material from any organism. A genetic material can be viral genomic DNA or RNA, nuclear genetic material, such as genomic DNA, or genetic material present in cell organelles, such as mitochondrial DNA or chloroplast DNA. It can also represent the genetic material coming from a natural or artificial mixture or a mixture of genetic material from several organisms.

A “target sequence” as used herein is a polynucleotide (e.g., as defined herein, including a DNA, RNA, or DNA/RNA hybrid, as well as modified forms thereof) that includes a “target site.” The terms “target site” is used to refer to a nucleic acid sequence present in a target genomic sequence (e.g., DNA or RNA in a host or pathogen) to which a primer and/or probe (e.g., any herein) will bind provided sufficient conditions (e.g., sufficient complementarity) for binding exist. Suitable DNA/RNA binding conditions include physiological conditions normally present in a cell. Other suitable DNA/RNA binding conditions (e.g., conditions in a cell-free system) are known in the art.

As used herein, sequences that are “sufficient complementarity” or “sufficiently complementary” refers to sequences that permit at least a portion of complementary sequences to anneal with each other.

The term “hybridizes with” when used with respect to two sequences indicates that the two sequences are sufficiently complementary to each other to allow nucleotide base pairing between the two sequences. As used herein, sequences that have “sufficient complementarity” or are “sufficiently complementary” permit at least a portion of complementary sequences to anneal with each other, such as, for example, at least about 50%, about 60% about 70%, about 80%, about 90%, about 95%, about 98%, about 99%, or about 99.9%. Sequences that hybridize with teach other can be perfectly complementary but can also have mismatches to a certain extent. Therefore, the sequences at the 5′ and 3′ ends of the forward and backward primers described herein may have a few mismatches with the corresponding target sequences at the 5′ and 3′ ends of the target nucleotide region as long as the forward and reverse primers can hybridize with the target sequences. Depending upon the stringency of hybridization, a mismatch of up to about 5% to 20% between the two complementary sequences would allow for hybridization between the two sequences. Typically, high stringency conditions have higher temperature and lower salt concentration, and low stringency conditions have lower temperature and higher salt concentration. High stringency conditions for hybridization are preferred.

“Hybridizing conditions” refer to conditions of temperature, pH, and concentrations of reactants that allow at least a portion of complementary sequences to anneal with each other. Conditions required to accomplish hybridization depend on the size of the oligonucleotides to be hybridized, the degree of complementarity between the oligonucleotides and the presence of other materials in the hybridization reaction admixture. The actual conditions necessary for each hybridization step are well known in the art or can be readily determined by a person of ordinary skill in the art. Typical hybridizing conditions include the use of solutions buffered to a pH from about 7 to about 8.5 and temperatures of from about 30° C. to about 80° C. Hybridization conditions may also include a buffer that is compatible, i.e., chemically inert, with respect to the oligonucleotides and other components, yet still allows for hybridization between complementary base pairs.

A “primer” is an oligonucleotide capable of initiating synthesis of nucleic acid sequence in a nucleic acid amplification reaction. A primer initiates nucleic acid amplification when placed under conditions in which synthesis is induced of a primer extension product that is complementary to a template nucleic acid strand. Such conditions include provision of appropriate nucleotides, an enzyme for polymerization such as a DNA polymerase, an appropriate buffer and a suitable temperature. Primers are synthesized based on the sequence of a target locus. For example, based on the sequence of a target locus and the sequences flanking the target locus, a skilled artisan can determine the sequence of a primer or a primer pair for amplification of the target locus.

A primer pair is a pair of oligonucleotides and designed to amplify a specific locus from a template nucleotide sequence material. Guidelines for designing a primer pair to amplify a specific locus to in a template genetic material are well known in the art.

As used herein, the phrases “operably linked” or “operatively linked” or “operatively associated with,” as used interchangeably, refers to a juxtaposition wherein the components so described are in a relationship permitting them to function in their intended manner. A first component can be operably linked to a second component by way of any useful bond (e.g., a covalent bond, a non-covalent bond, and/or linked via van der Waals forces, hydrogen bonds, and/or other intermolecular forces, such as those including a π-π interaction, a salt bridge, or a cation-it interaction) or any useful linker (e.g., any herein).

Throughout this disclosure, different sequences are described by specific nomenclature, for example, a primer binding sequence, primer sequence, ID region, amplifier sequence, and target sequence. When such nomenclature is used, it is understood that the identified sequence is substantially identical or substantially reverse complementary to at least a part of the corresponding sequence. For example, “a primer sequence” describes a sequence that is substantially identical to at least a part of the primer sequence or substantially reverse complementary to at least a part of the primer sequence. This is because when a captured target genomic region is converted into a double stranded form comprising the primer binding sequence, the double stranded target genomic region can be sequenced using a primer having a sequence that substantially identical or substantially reverse complementary to at least a part of primer binding sequence. Thus, the nomenclature is used herein to simplify the description of different polynucleotides and parts of polynucleotides used in the methods disclosed here; however, a person of ordinary skill in the art would recognize that appropriate substantially identical or substantially reverse complementary sequences to at least a part of the corresponding sequences could be used to practice the methods disclosed herein.

Also, two sequences that correspond to each other, for example, a primer binding sequence and a primer sequence, have at least 90% sequence identity, preferably, at least 95% sequence identity, even more preferably, at least 97% sequence identify, and most preferably, at least 99% sequence identity, over at least 70%, preferably, at least 80%, even more preferably, at least 90%, and most preferably, at least 95% of the sequences. Alternatively, two sequences that correspond to each other are reverse complementary to each other and have at least 90% perfect matches, preferably, at least 95% perfect matches, even more preferably, at least 97% perfect matches, and most preferably, at least 99% perfect matches in the reverse complementary sequences, over at least 70%, preferably, at least 80%, even more preferably, at least 90%, and most preferably, at least 95% of the sequences. Thus, two sequences that correspond to each other can hybridize with each other or hybridize with a common reference sequence over at least 70%, preferably, at least 80%, even more preferably, at least 90%, and most preferably, at least 95% of the sequences. Preferably, two sequences that correspond to each other are 100% identical over the entire length of the two sequences or 100% reverse complementary over the entire length of the two sequences.

As used herein, the terms “identical” or percent “identity”, in the context of describing two or more polynucleotide sequences, refer to two or more sequences or subsequences that are the same or have a specified percentage of nucleotides that are the same (for example, a nucleotide probe used in the method of this invention has at least 70% sequence identity, preferably 80%, 85%, 90%, 91%, 92%, 93%, 94%, 95%, 96%, 97%, 98%, 99%, or 100% identity, to a target sequence or complementary sequence thereof), when compared and aligned for maximum correspondence over a comparison window, or designated region as measured using one of the following sequence comparison algorithms or by manual alignment and visual inspection. Such sequences are then said to be “substantially identical”. With regard to polynucleotide sequences, this definition also refers to the complement of a test sequence.

As used herein, the term “multiplexing” refers to a process in which multiple samples are pooled together for signal readout and processing, such as, for example, mixing sequences from multiple subjects into one pool for sequence amplification and/or screening of samples; or, in another example, generating a mixture of sequences derived from DNA and RNA for amplification or sequencing together.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims.

### Primer and Probe Design and Detection

The methods disclosed herein provide for adding distinct primers and/or probes to one or more target nucleotide sequences in two or more samples, pooling the samples, and amplifying the one or more target nucleotide sequences in the pooled sample. The methods comprise providing two or more samples than can each contain none or one or more target nucleotide sequences and two or more distinct primers that each contain the target-specific region complementary to the target nucleotide sequence of interest, the ID region that provides a unique identifier sequence and can be differentiated by its melting temperature by, for example, altering the nucleotides of the sequence and/or the length of the sequence, and the amplifier sequence for the subsequent PCR primer binding region. In certain embodiments, each ID region can be designed to be orthogonal to each other (non-interacting) to eliminate primer dimerization, and the amplifier sequence of each ID-Primer can also be also designed to be different to eliminate the bias from the varying target concentration in different samples.

In certain embodiments, a unique ID-Primer is added to each sample after RNA extraction from the sample. If the sample contains RNA, the ID Primer can act as a reverse transcription primer, thus generating a cDNA-RNA duplex upon the addition of reverse transcriptase without Ribonuclease H (RNase H) activity. If the sample contains DNA, the primer can remain as single-stranded DNA without the RNA target. Then, an exonuclease can be added to each sample, such as, for example, exonuclease I. Since an exonuclease can only degrade single stranded DNA (from 3′ to 5′ end), it will selectively digest all unused ID-Primers and only the cDNA-RNA duplexes remain intact. Therefore, each sample that is positive for the RNA target will be labeled with a special tag and can then be differentiated after being pooled one or more other samples.

In alternative embodiments, a unique ID-Primer is added to each sample after RNA extraction from the sample through the specific hydrogen bonding patterns of nucleobases. The secondary structures of single strand RNA templates are loosened by heating to about 65° C. and then the ID-Primers can be annealed to the complementary RNA strands at about 4° C. to about 30° C. After annealing, an exonuclease can be added to each sample, such as, for example, exonuclease I, to digest the unhybridized ID-Primers. Therefore, each sample that is positive for the RNA target will be labeled with a special tag and can then be differentiated after being pooled with one or more other samples.

In an alternative embodiment, sample preparation and adding ID-Primers can be combined into one step which involves the extraction-free sample preparation method for raw RNA samples. In certain embodiments, any commercially available lysis buffer for RNA extraction can be added to the sample. In certain embodiments, a single-stranded oligonucleotide probe to capture target nucleotide sequences (i.e., the capture strand) can be created in which the capture stranded is labelled, with, for example, a biotin label and attached to the streptavidin coated magnetic bead (MB) through the streptavidin-biotin interaction. Then the streptavidin coated magnetic beads linked with capture strands and ID-Primers can be introduced to the mixtures, and the RNA targets will be specifically hybridized with the capture strand. At the same time, the ID-Primers can also bind to different regions of RNA targets. After that, the unbound primers and impurities can be washed away without the use of an exonuclease, and only the DNA-RNA duplexes are preserved. Next, all the samples will be pooled together and then go through the reverse transcription (RT) and detection steps. During the RT step, the ID-Primers will act as RT primers and be extended to generate ID-Samples. At the same time, the RNA capture strand with MB hybridized with RNA target will be released from the DNA-RNA duplex by the displacement activity of reverse transcriptase. Therefore, the MB can be separated by magnetic field. After magnetic separation, the separated ID-Samples can be amplified by PCR and detected by size-separation tools, multiplexed melting curve analysis or electrochemical station.

In certain embodiments, primers can be designed to hybridize or ligate to the target nucleic acid sequence, or portions thereof, as well as amplicons derived from the target nucleic acid sequence. In certain embodiments, primers can be designed to prime a reverse transcriptase and/or polymerase. In certain embodiments, the complementary nucleotide segment of the primer or probe is 1, 2, 3, 4, 5, 10, 11, 12, 13, 14, 15, 16, 17, 18, 19, 20, 21, 22, 23, 24, 25, 30, 40, 50, or 100 base pairs long, or longer. Furthermore, the primer (e.g., any herein, such as an ID-Primer, forward or reverse primer) can be labeled with a fluorescent label (e.g., for use with a quencher probe), electroactive label, or can be unlabeled. The concentration of the primer and probes can be optimized to promote the amplification or reverse transcriptase reaction.

In certain embodiments, the primers and probes herein can include any useful label, including fluorescent labels and quencher labels at any useful position in the nucleic acid sequence, such as, for example at the 3′- and/or 5′-terminus. Exemplary fluorescent labels include a quantum dot, a fluorophore. Examples of fluorescence labels for use in this method includes fluorescein, 6-FAM™ (Applied Biosystems, Carlsbad, Calif.), TET™ (Applied Biosystems, Carlsbad, Calif.), VIC™ (Applied Biosystems, Carlsbad, Calif.), MAX, HEX™ (Applied Biosystems, Carlsbad, Calif.), TYE™ (ThermoFisher Scientific, Waltham, Mass.), TYE665, TYE705, TEX, JOE, Cy™ (Amersham Biosciences, Piscataway, N.J.) dyes (Cy2, Cy3, Cy3B, Cy3.5, Cy5, Cy5.5, Cy7), Texas Red® (Molecular Probes, Inc., Eugene, Oreg.), Texas Red-X, AlexaFluor® (Molecular Probes, Inc., Eugene, Oreg.) dyes (AlexaFluor 350, AlexaFluor 405, AlexaFluor 430, AlexaFluor 488, AlexaFluor 500, AlexaFluor 532, AlexaFluor 546, AlexaFluor 568, AlexaFluor 594, AlexaFluor 610, AlexaFluor 633, AlexaFluor 647, AlexaFluor 660, AlexaFluor 680, AlexaFluor 700, AlexaFluor 750), DyLight™ (ThermoFisher Scientific, Waltham, Mass.) dyes (DyLight 350, DyLight 405, DyLight 488, DyLight 549, DyLight 594, DyLight 633, DyLight 649, DyLight 755), ATTO™ (ATTO-TEC GmbH, Siegen, Germany) dyes (ATTO 390, ATTO 425, ATTO 465, ATTO 488, ATTO 495, ATTO 520, ATTO 532, ATTO 550, ATTO 565, ATTO Rhol01, ATTO 590, ATTO 594, ATTO 610, ATTO 620, ATTO 633, ATTO 635, ATTO 637, ATTO 647, ATTO 647N, ATTO 655, ATTO 665, ATTO 680, ATTO 700, ATTO 725, ATTO 740), BODIPY® (Molecular Probes, Inc., Eugene, Oreg.) dyes (BODIPY FL, BODIPY R6G, BODIPY TMR, BOPDIPY 530/550, BODIPY 558/568, BODIPY 564/570, BODIPY 576/589, BODIPY 581/591, BODIPY 630/650, BODIPY 650/665), HiLyte Fluor™ (AnaSpec, Fremont, Calif.) dyes (HiLyte Fluor 488, HiLyte Fluor 555, HiLyte Fluor 594, HiLyte Fluor 647, HiLyte Fluor 680, HiLyte Fluor 750), AMCA, AMCA-S, Cascade® Blue (Molecular Probes, Inc., Eugene, Oreg.), Cascade Yellow, Coumarin, Hydroxycoumarin, Rhodamine Green™-X (Molecular Probes, Inc., Eugene, Oreg.), Rhodamine Red™-X (Molecular Probes, Inc., Eugene, Oreg.), Rhodamine 6G, TMR, TAMRA™ (Applied Biosystems, Carlsbad, Calif.), 5-TAMRA, ROX™ (Applied Biosystems, Carlsbad, Calif.), Oregon Green® (Life Technologies, Grand Island, N.Y.), Oregon Green 500, IRDye® 700 (Li-Cor Biosciences, Lincoln, Nebr.), IRDye 800, WeIIRED D2, WeIIRED D3, WeIIRED D4, and Lightcycler® 640 (Roche Diagnostics GmbH, Mannheim, Germany). In some embodiments, bright fluorophores with extinction coefficients >50,000 M⁻¹ cm⁻¹ and appropriate spectral matching with the fluorescence detection channels can be used.

In certain embodiments, a fluorescently labeled primer or probe is included in a reaction mixture and a fluorescently labeled reaction product is produced. Fluorophores used as labels to generate a fluorescently labeled primer and/or probes included in embodiments of methods and compositions of the present invention can be any of numerous fluorophores including, but not limited to, 4-acetamido-4′-isothiocyanatostilbene-2,2′ disulfonic acid; acridine and derivatives such as acridine and acridine isothiocyanate; 4-amino-N-[3-vinylsulfonyl)phenyl]naphthalimide-3,5 disulfonate, Lucifer Yellow VS; N-(4-anilino-1-naphthyl)maleimide; anthranilamide, Brilliant Yellow; BIODIPY fluorophores (4,4-difluoro-4-bora-3a,4a-diaza-s-indacenes); coumarin and derivatives such as coumarin, 7-amino-4-methylcoumarin (AMC, Coumarin 120), 7-amino-4-trifluoromethylcoumarin (Coumaran 151); cyanosine; DAPDXYL sulfonyl chloride; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-4′-dimethylaminophenylazo)benzoic acid (DABCYL); 4-dimethylaminophenylazophenyl-4′-isothiocyanate (DABITC); EDANS (5-[(2-aminoethyl)amino]naphthalene-1-sulfonic acid), eosin and derivatives such as eosin isothiocyanate; erythrosin and derivatives such as erythrosin B and erythrosin isothiocyanate; ethidium such as ethidium bromide; fluorescein and derivatives such as 5-carboxyfluorescein (FAM), hexachlorofluorescenin, 5-(4,6-dichlorotriazin-2-yl)aminofluorescein (DTAF), 2′,7′-dimethoxy-4′,5′-dichloro-6-carboxyfluorescein (JOE) and fluorescein isothiocyanate (FITC); fluorescamine; green fluorescent protein and derivatives such as EBFP, EBFP2, ECFP, and YFP; IAEDANS (5-({2-[(iodoacetyl)amino]ethyl} amino)naphthalene-1-sulfonic acid), Malachite Green isothiocyanate; 4-methylumbelliferone; orthocresolphthalein; nitrotyrosine; pararosaniline; Phenol Red; B-phycoerytnin; o-phthaldialdehyde; pyrene and derivatives such as pyrene butyrate, 1-pyrenesulfonyl chloride and succinimidyl 1-pyrene butyrate; QSY 7; QSY 9; Reactive Red 4 (Cibacron® Brilliant Red 3B-A); rhodamine and derivatives such as 6-carboxy-X-rhodamine (ROX), 6-carboxyrhodamine (Rhodamine 6G), rhodamine isothiocyanate, lissamine rhodamine B sulfonyl chloride, rhodamine B, rhodamine 123, sulforhodamine B, sulforhodamine 101 and sulfonyl chloride derivative of sulforhodamine 101 (Texas Red); N,N,N′,N-tetramethyl-carboxyrhodamine (TAMRA); tetramethyl rhodamine; tetramethyl rhodamine isothiocyanate (TRITC); riboflavin; rosolic acid and terbium chelate derivatives. In certain embodiments, the concentration of the fluorescent probe or primer in the compositions and method of use is about 0.01 μM to about 100 μM, about 0.1 μM to about 100 μM, about 0.1 M to about 50 μM, about 0.1 μM to about 10 μM, or about 1 μM to about 10 μM. In certain embodiments, the concentration of the fluorescent probe or primer is about 0.01 μM, 0.1 μM, 1 M, 1.1 μM, 1.2 μM, 1.3 μM, 1.4 μM, 1.5 μM, 1.6 μM, 1.7 μM, 1.8 μM, 1.9 μM, 2 μM, 2.5 μM, or 5 μM.

Exemplary quencher labels include a fluorophore, a quantum dot, a metal nanoparticle, etc.). Suitable quenchers include Black Hole Quencher®-1 (Biosearch Technologies, Novato, Calif.), BHQ-2, Dabcyl, Iowa Black® FQ (Integrated DNA Technologies, Coralville, Iowa), IowaBlack RQ, QXL™ (AnaSpec, Fremont, Calif.), QSY 7, QSY 9, QSY 21, QSY 35, IRDye QC, BBQ-650, Atto 540Q, Atto 575Q, Atto 575Q, MGB 3′ CDPI3, MGB-5′ CDPI3, and an inverted dT. In one instance, the term “quencher” refers to a substance which reduces emission from a fluorescent donor when in proximity to the donor. In preferred embodiments, the quencher is within 1, 2, 3, 4, 5, 6, 7, 8, 9, 10, 11, 12, 13, 14, 15, 16, 17, 18, 19, 20, 25, or 30 nucleotide bases of the fluorescent label. In certain embodiments, the quencher can be added to the 3′ end of the probe or primer. Fluorescence is quenched when the fluorescence emitted from the fluorophore is detectably reduced, such as reduced by 10%, 20%, 30%, 40%, 50%, 60%, 70%, 80%, 90%, 95%, 99% or more.

In certain embodiments, an electroactively labeled primer or probe is included in a reaction mixture and an electroactively labeled reaction product is produced. Electroactive reporters can be used as labels to generate an electroactively labeled probe or primer included in embodiments of methods and compositions of the present invention can be any of numerous Electroactive reporters including, but not limited to methylene blue, Anthraquinone, Ru(bpy)2dppz2+, Ru(phen)2dppz2+, Ferrocene derivative, hematoxylin, magnetic bead, QD, biotin-advinHRP, nano composite and ferrocene. In certain embodiments, the concentration of the electroactive probe or primer in the compositions and method of use is about 0.01 μM to about 100 μM, about 0.1 μM to about 100 μM, about 0.1 μM to about 50 μM, about 0.1 μM to about 10 μM, or about 1 μM to about 10 μM. In certain embodiments, the concentration of the electroactive probe or primer is about 0.01 μM, 0.1 μM, 1 μM, 1.1 μM, 1.2 μM, 1.3 μM, 1.4 μM, 1.5 μM, 1.6 μM, 1.7 μM, 1.8 μM, 1.9 μM, 2 μM, 2.5 μM, or 5 μM.

Other labels can be used in the subject in invention, including those that permit colorimetric, and chemiluminescent or fluorescent detection. For example, biotin or digoxin are well-known in the art and can be used in conjunction with, anti-digoxin antibodies and streptavidin that are couple to alkaline phosphatase, horseradish peroxidase, or fluorescein or rhodamine (as described above), to permit colorimetric, and chemiluminescent or fluorescent detection.

Any detection method or system operable to detect a labeled reaction product can be used in methods according to embodiments of the present invention and such appropriate detection methods and systems are well-known in the art. In certain embodiments, the target nucleotide sequences can be detected directly or detected indirectly via a released probe. Detection of the amplified nucleic acid may be performed by a method selected from gel electrophoresis, a fragment analyzer, or a bioanalyzer, intercalating dye detection, PCR, real-time PCR, fluorescence, Fluorescence Resonance Energy Transfer (FRET), mass spectrometry, lateral flow assay, colorimetric assays, and CRISPR-based detection system. A signal from the fluorescently labeled reaction product is detected, for instance, using a photodiodes.

In certain embodiments, a probe or primer in the reaction can have at least one, two, three, four, or more sequences that facilitate further processing or detection. Such sequences include restriction primer binding sites, particularly amplifier sequence sites. In preferred embodiments, at least one of the amplifier sites has a nucleotide substitution, addition, or deletion of at least one, two, three, four, five, six, or more nucleotides when compared to a nucleotide sequence that can be recognized by an endonuclease. In preferred embodiments, the probe or primer has one amplifier site, preferably an amplifier site operably linked to a label, has that can be recognized by an DNA primer for use in PCR.

In certain embodiments, additional sequences including DNA or RNA bases can be added, deleted, substituted or modified in the primers and probe to confer advantageous properties. The 5′ or 3′ regions can be modified, specifically one, two, three, four, five, six, seven, eight, nine, ten, or more DNA or RNA bases can be added, deleted, or substituted. In preferred embodiments, a 3′ inverted dT dimer is added to a probe to prevent extension of by a polymerase.

In certain embodiments, a sample specific primer and/or probe can be used. The primer and/or probe can have a distinct label, distinct melting temperature, distinct sequence, distinct sequence length, or any combination thereof when compared to the primer and/or probe used for each subsequent sample.

### Detecting Target Nucleotide Sequences in Pooled Samples

In preferred embodiments, the presence of a target nucleic acid is determined directly via fragment length identification or indirectly via a probe from pooled samples of either reverse transcribed target RNA (cDNA) or target DNA, as described above. In certain embodiments, in the presence of a target nucleotide sequence, the probe and/or label is released after amplification of the target sequence. The detection of this label can be performed in using a variety of well-known methods. Examples of detection methods include electroactive assay or fluorescent assay. Upon a polymerase chain reaction assay, a fluorophore or electroactive reporter can be released.

In certain embodiments, the target sequences can be amplified via asymmetric PCR to generate single-stranded amplicons from the cDNA in the pooled sample using a universal forward primer and two or more distinct reverse primer that is specific to each ID-Primer, specifically the amplifier region of the ID-Primer. The abundant universal forward primer will hybridize with the sequence of target without ID-Primers to produce complementary sequences of ID-samples in the format of single strand, and distinct reverse primers will bind with the complementary sequences of ID-Primers part to assist these amplification reactions. After that, the generated single strand amplicons will be used as detection targets in the following analysis. A probe set comprising 1, 2, 3, 4, 5, 6, 7, 8, 9, 10, or more (up to the total number of samples that have been pooled) probes with the fluorophore (F probe; complementary to the ID region of the ID-Primer) and a probe with the quencher (Q probe; complementary to the cDNA region adjacent to the F probe-binding region). The Q probe can comprise nucleotides that enable the probe to have a melting temperature (Tₘ) value of about 70° C. to about 80° C. or a Tₘ at least higher by about 5° C., about 6° C., about 7° C., about 8° C., about 9° C., about 10° C., or more than the melting temperature of each of the F probes. The F and Q probes will hybridize to their corresponding sites at around 45° C., and at this point, the fluorescence signal is quenched. When the temperature reaches the Tₘ value of the F probe, the F probe will be released and would yield a sharp increase in fluorescence signal that can be detected via melting curve analysis. By designing different fluorophores and Tₘ values of the F-probe, the melting curve peak can be correlated to the presence of the specific tagged cDNA target. In an alternative embodiment, universal F and Q probes can be used. The F probe can be designed to be fully complementary to the ID region of a first sample. The F probe can be designed so that as it hybridizes with additional samples, but overhanging ends are created upon hybridization. For example, upon hybridization with a second sample, it can form a 2-nucleotide overhanging end and a 4-nucleotide overhanging end with a 3^{rd} sample. The overhanging ends can decrease the Tₘ value during the melting curve analysis.

In an alternative embodiment, a probe set comprising 1, 2, 3, 4, 5, 6, 7, 8, 9, 10, or more (up to the total number of samples that have been pooled) probes with a fluorophore and a quencher (e.g., TaqMan probe) can used to identify the presence of target sequences after sample pooling. The target sequences in the pooled samples can be amplified via PCR to generate amplicons from the cDNA using a universal forward primer and two or more distinct reverse primers that are each specific to an ID-Primer, specifically the amplifier region of the ID-Primer. During the PCR amplification process, the probes labeled with distinct fluorophores will hybridize with the targets and can be hydrolyzed by the exonuclease activity of the polymerase, providing real time fluorescent signals. If the target nucleotides exist, there will be exponential growth. According to the cycle threshold value, this assay can achieve quantitative and qualitative analysis of grouped samples.

In an alternative embodiment, after sample pooling, the first step involves the PCR or isothermal amplification of the pooled samples, using a universal forward primer and two or more distinct reverse primer that are each specific to an ID-Primer, specifically the amplifier region of the ID-Primer, to produce double stranded amplicons with unique lengths. The lengths of the amplicons are determined by the length of the ID-Primers initially added to the target sequence. Each ID-Primer can have distinct lengths. Thus, in this step, the initial RNA/DNA duplex will be converted to a double-stranded DNA molecule. This product is then detected using the fragment analyzer, gel electrophoresis, or bioanalyzer. By comparing the bands in a sample to the DNA ladder, the positive samples can be inferred by the approximate size of products which amplify from RNA/DNA duplexes. If no band is detected, then all individual samples are declared negative, but if the band is present in the expected position, the length of the product will reveal which sample is positive.

In an alternative embodiment, the methods involve mixing the pooled samples with amplification primers, a universal forward primer and two or more distinct reverse primers that are specific to each ID-Primer, a DNA polymerase with exonuclease activity, and homologous probes that are labeled with electroactive reporters with non-overlapping redox potentials, such as, for example, ferrocene, methylene blue, or anthraquinone. Because the DNA polymerase has exonuclease property, the electrochemical probes that hybridized with the target sequences will be hydrolyzed during the extension step. Thus, the electroactive reporters with short mononucleotides can be released, which can generate a high current peak at certain potential. After amplification, the end point electrochemical test will be carried out. As a result, the sample with a target sequence can be differentiated from the target-free sample by the existence of high current peak at certain potential.

A signal from the electroactively labeled reaction can be detected, for instance, using a carbon screen-printed electrode or any other electrochemical label-based bioassay. Two types of using a carbon screen printed electrode can be used in the subject methods. The first type includes running the reaction on an incubator or PCR thermocycler, and, after that, dropping an appropriate volume of the reaction solution onto the electrode surface, ensuring that all the electrodes (working electrode, counter electrode and reference electrodes) are well covered by the reaction solution. Then, the electrochemical signal of the no template control (NTC) and the sample using electrochemical station can be recorded. The second type includes conducting the reaction into an electrochemical detection chamber with the carbon screen parented electrode under the cell. Then, the electrochemical signal changes using electrochemical station in real-time can be recorded.

### Kits

In certain embodiments, the present compositions and methods of use can further be provided in a kit. The kit can include one or more of the following: one or more primers (ID-Primers; forward and reverse primers), one or more probes (F probe, Q probe), other reagents (e.g., any described herein, such as enzymes, buffer, nucleotides, or enhancing agents), particularly reagents that one skilled in the art would recognize as necessary or beneficial for reverse transcription, PCR, Loop-mediated isothermal Amplification (LAMP), Recombinase Polymerase Amplification (RPA), Rolling Circle Amplification (RCA), or Nucleic Acid Sequence-based Amplification (NASBA), and instructions for use (e.g., such as those including any method described herein). Each component of the kit can be packaged separately or together. In one instance, the components are packaged together to allow for a single chamber or single test tube reaction.

### Enzymes

In certain embodiments, one or more enzymes can be used, including a plurality of polymerases and exonucleases. If the target nucleic acid includes a RNA sequence, or a portion of an RNA sequence, then a reverse transcriptase can be employed to reverse transcribe the RNA target into a DNA (e.g., cDNA) sequence.

In certain embodiments, a DNA polymerase can be used, including a DNA polymerase with exonuclease activity. Exemplary polymerase enzymes include Bst DNA polymerase (including Bst 3.0; New England BioLabs, Inc., Ipswich, Mass.), Bca (exo-)DNA polymerase, DNA polymerase I Klenow fragment, Vent DNA polymerase, Vent (exo-)DNA polymerase (Vent DNA polymerase deficient in exonuclease activity), Vent™ DNA polymerase, 9° N™ polymerase, Deep Vent DNA polymerase, Deep Vent(exo-)DNA polymerase (Deep Vent DNA polymerase deficient in exonuclease activity), 129 phage DNA polymerase, MS-2 phage DNA polymerase, Z-Taq DNA polymerase (Takara Shuzo Co., Ltd.), Taq polymerase, and KOD DNA polymerase (Toyobo Co., Ltd.), as well as variants thereof.

Exemplary exonuclease enzymes include exonuclease I.

Reverse transcriptases useful in the present invention can be any polymerase that exhibits reverse transcriptase activity. Several reverse transcriptases are known in the art and are commercially available (e.g., from Boehringer Mannheim Corp., Indianapolis, Ind.; Life Technologies, Inc., Rockville, Md.; New England Biolabs, Inc., Beverley, Mass.; Perkin Elmer Corp., Norwalk, Conn.; Pharmacia LKB Biotechnology, Inc., Piscataway, N.J.; Qiagen, Inc., Valencia, Calif.; Stratagene, La Jolla, Calif.). In some embodiments, the reverse transcriptase can be Avian Myeloblastosis Virus reverse transcriptase (AMV-RT), Moloney Murine Leukemia Virus reverse transcriptase (M-MLV-RT), Human Immunovirus reverse transcriptase (HIV-RT), EIAV-RT, RAV2-RT, C. hydrogenoformans DNA Polymerase, rTth DNA polymerase, SUPERSCRIPT I, SUPERSCRIPT II, and mutants, variants and derivatives thereof. In preferred embodiments, the reverse transcriptase is an RNase H minus (i.e., lacks RNase activity) reverse transcriptase. It is to be understood that a variety of reverse transcriptases can be used in the present invention, including reverse transcriptases not specifically disclosed above, without departing from the scope or preferred embodiments disclosed herein.

### Nucleotide Bases

Nucleotide bases useful in the present invention can be any nucleotide useful in the polymerization of a nucleic acid. Nucleotides can be naturally occurring, unusual, modified, derivative, or artificial. Nucleotides can be unlabeled, or detectably labeled by methods known in the art (e.g., using radioisotopes, vitamins, fluorescent or chemiluminescent moieties, dioxigenin). Preferably the nucleotides are deoxynucleoside triphosphates, dNTPs (e.g., dATP, dCTP, dGTP, dTTP, dITP, dUTP, α-thio-dNITs, biotin-dUTP, fluorescein-dUTP, digoxigenin-dUTP, 7-deaza-dGTP). dNTPs are also well known in the art and are commercially available from venders (e.g., from Boehringer Mannheim Corp., Indianapolis, Ind.; New England Biolabs, Inc., Beverley, Mass.; Pharmacia LKB Biotechnology, Inc., Piscataway, N.J.).

The nucleotides of the present invention can be present in any concentration. In some embodiments, the nucleotides are present in an amount from about 0.001 μM to about 40 PM, about 0.005 μM to about 20 μM, or, preferably, about 0.01 μM to about 4 μM. One of skill in the art will appreciate that other concentrations of nucleotides are useful in the present invention.

### Buffering Agents and Salts

Buffering agents and salts useful in the present invention provide appropriate stable pH and ionic conditions for nuclease activity and/or nucleic acid synthesis, e.g., for reverse transcriptase and DNA polymerase activity. A wide variety of buffers and salt solutions and modified buffers are known in the art that can be useful in the present invention, including agents not specifically disclosed herein. Preferred buffering agents include, but are not limited to, Tris-HCl, NaCl, MgCl₂, and BSA. Preferred salt solutions include, but are not limited to solutions of, potassium acetate, potassium sulfate, potassium chloride, ammonium sulfate, ammonium chloride, ammonium acetate, magnesium chloride, magnesium acetate, magnesium sulfate, manganese chloride, manganese acetate, manganese sulfate, sodium chloride, sodium acetate, lithium chloride, and lithium acetate.

The buffering agents of the present invention can be present in any concentration. In some embodiments, the buffering agent is present in an amount from about 0.01 mM to about 4000 mM, about 0.05 mM to about 2000 mM, or, preferably, about 0.1 mM to about 400 mM. One of skill in the art will appreciate that other concentrations of buffer are useful in the present invention.

### Methods of Use

In certain embodiments, the present probes, primers, assays, and methods can be used to detecting any target of interest. In particular, the probes, primers, assays, and methods allow for a sample-specific testing without the need for retesting. In some embodiments, the compositions and methods can be configured for sensing a nucleic acid (e.g., RNA and/or DNA), as well as for detecting a pathogen (e.g., a viral pathogen, such as any herein).

The present compositions and methods can be used to detect any useful targets (e.g., a target nucleic acid or a nucleic acid sequence derived from the target or identifiable as the target). Exemplary targets include an RNA virus, such as Arenaviridae (e.g., Machupo virus), Bunyaviridae (e.g., Hantavirus or Rift Valley fever virus), Coronaviridae (e.g., SARS-CoV, MERS-CoV, SARS-CoV-2), Orthomyxoviridae (e.g., influenza viruses), Filoviridae (e.g., Ebola virus and Marburg virus), Flaviviridae (e.g., Japanese encephalitis virus and Yellow fever virus), Paramyxoviridae (e.g., respiratory syncytial virus, measles virus, mumps virus, or parainfluenza virus), Picornaviridae (e.g., polioviruses), Reoviridae (e.g., rotaviruses), Retroviridae (e.g., human T cell lymphotropic viruses (HTLV) and human immunodeficiency viruses (HIV)), Rhabdoviridae (e.g., rabies virus), and Togaviridae (e.g., encephalitis viruses, yellow fever virus, and rubella virus)); a pathogen; an environmental contaminant; a water additive; an agricultural marker; a nucleic acid (e.g., oligonucleotides, polynucleotides, nucleotides, nucleosides, or molecules of RNA, a viral genome, a primer, or a gene of any useful pathogen, such as those described herein); or a genetic modification (e.g., antibiotic resistance marker gene). Targets also include food-borne pathogens, such as Norovirus (e.g., Norwalk virus); and weaponized pathogens, such as, Filoviridae (e.g., Ebola virus and Marburg virus), Arenaviridae (e.g., Lassa virus and Machupo virus), Alphavirus (e.g., Venezuelan equine encephalitis virus, eastern equine encephalitis virus, or western equine encephalitis virus), Henipavirus (e.g., Nipah virus), Bunyaviridae (e.g., Hantavirus or Rift Valley fever virus), and Flaviviridae (e.g., Japanese encephalitis virus and Yellow fever virus).

The test sample can include any useful sample, such as a virus, a cell, tissue, a fluid, a swab, a biological sample (e.g., blood, serum, plasma, saliva, etc.), a plant, an environmental sample (e.g., air, soil, and/or water), etc.

### Materials and Methods

### Preparation of RNA Samples and DNA Oligonucleotides

The synthetic SARS-CoV-2 RNA target was purchased from European Commission Joint Research Centre and the synthetic Influenza A (H1N1) RNA sample for selectivity test was obtained from the GeneWell (Shenzhen, China). All RNA samples were serially diluted using 1×TE buffer (10 mM Tris, 1 mM EDTA, pH=8.0, Invitrogen) and aliquoted, and then stored at −80° C. for further use. Synthetic oligonucleotides were purchased from Generay (Shanghai, China) with HPLC purification. All oligonucleotides were re-suspended in 1×TE buffer to 100 nM and stored at 4° C. upon arrival. Their concentrations were measured using Nanovue Plus spectrophotometer (GE Healthcare). The sequences of all the RNA samples and DNA oligos used are listed in Table 1.

TABLE 1
detail sequences of target RNA samples, primers and probes.

| Name | Detailed sequence (5′ to 3′) |
|---|---|
| Synthetic RNA(EURO-019) | GGGAGACGAAUUGGGCCCUCUAGAUGCAUGCUCGAGC |
| | GGCCGCCAGUGUGAUGGAUAUCUGCAGAAUUCGCCCU |
| | UAUUCAAGUAUUGAGUGAAAUGGUCAUGUGUGGCGG |
| | UUCACUAUAUGUUAAACCAGGUGGAACCUCAUCAGGA |
| | GAUGCCACAACUGCUUAUGCUAAUAGUGUUUUUAACA |
| | UUUGUCAAGCUGUCCGGAAGAGACAGGUACGUUAAUA |
| | GUUAAUAGCGUACUUCUUUUUCUUGCUUUCGUGGUA |
| | UUCUUGCUAGUUACACUAGCCAUCCUUACUGCGCUUC |
| | GAUUGUGUGCGUACUGCUGCAAUAUUGUUAACGUAUA |
| | AUGGACCCCAAAAUCAGCGAAAUGCACCCCGCAUUAC |
| | GUUUGGUGGACCCUCAGAUUCAACUGGCAGUAACCAG |
| | AAUGGAGAACGCAUUGCAACUGAGGGAGCCUUGAAUA |
| | CACCAAAAGAUCACAUUGGCACCCGCAAUCCUGCUAAC |
| | AAUGCUGCAAUCGUGCUACAACUUCCUCAAGGAAAUU |
| | UUGGGGACCAGGAACUAAUCAGACAAGGAACUGAUUA |
| | CAAACAUUGGCCGCAAAUUGCACAAUUUGCCCCCAGCG |
| | CUUCAGCGUUCUUCGGAAUGUCGCGCAUUGGCAUGGA |
| | AGUCACACCUUCGGGAACGUGGUUGACCUACACAGGU |
| | GCCAUCAAAUUGGAGUGUGACAUACCCAUUGGUGCAG |
| | GUAUAUGCGCUAGUUAUCAGACUCAGACUAAUUCUCC |
| | UCGGCGGGCACGUAGUGUAGCUAGUCAACCUGCUUUU |
| | GCUCGCUUGGAUCCGAAUUCAAAGGUGAAAUUGUUAU |
| | CCGCUCACAAUUCCACACAACAUACGAGCCGGAAGCAU |
| | AAAGUGUAAAGCCUGGGGUGCCUAAUGA (SEQ ID NO: |
| | 1) |
| PCR universal Forward | CGTATAATGGACCCCAAAATCAGCG (SEQ ID NO: 2) |
| primer | |
| Multiplex FAM Tag1 | GCAGAGTCGGCCTACAGGTTTATGTATCATAAGTCAAATG |
| | TCTGGTTACTGCCAGTTGAAT (SEQ ID NO: 3) |
| Multiplex FAM Tag2 | CTCACTGCCTACTACTCCACATAGCAACTGTAAGTTTTATG |
| | GCCTTCTCTTCTGGTTACTGCCAGTTGAAT (SEQ ID NO: 4) |
| Multiplex FAM Tag3 | CTCTCAACCTCCACCCCTTCAGTCTGCTAAGGTCATACGTC |
| | CCCTTGGCTCTCTGGTTACTGCCAGTTGAAT (SEQ ID NO: |
| | 5) |
| Multiplex HEX Tag4 | CCATTAGAACCTAAGCTACTCCACGCTATGTATCATAAGT |
| | CAAATGTCTGGTTACTGCCAGTTGAAT (SEQ ID NO: 6) |
| Multiplex HEX Tag5 | CGAGAGCCAGGTAACGAATGGTCATGTTGTTAAGGAGCG |
| | AATTAAATCTCTGGTTACTGCCAGTTGAAT (SEQ ID NO: |
| | 7) |
| Tagl Reverse Primer | GCAGAGTCGGCCTACAGGTTT (SEQ ID NO: 8) |
| Tag2 Reverse Primer | CTCACTGCCTACTACTCCACATAGCA (SEQ ID NO: 9) |
| Tag3 Reverse Primer | CTCTCAACCTCCACCCCTTCA (SEQ ID NO: 10) |
| Tag4 Reverse Primer | CCATTAGAACCTAAGCTACTCCACGCT (SEQ ID NO: 11) |
| Tag5 Reverse Primer | CGAGAGCCAGGTAACGAATGGT (SEQ ID NO: 12) |
| Fluorophore strand 1 | ATGTATCATAAGTCAAATG-FAM (SEQ ID NO: 13) |
| Fluorophore strand 2 | ACTGTAAGTTTTATGGCCTTCTCT-FAM (SEQ ID NO: 14) |
| Fluorophore strand 3 | GTCTGCTAAGGTCATACGTCCCCTTGGCTC-FAM (SEQ ID |
| | NO: 15) |
| Fluorophore strand 4 | ATGTATCATAAGTCAAATG-HEX (SEQ ID NO: 16) |
| Fluorophore strand 5 | CATGTTGTTAAGGAGCGAATTAAATC-HEX (SEQ ID NO: |
| | 17) |
| Quencher strand T | BHQ2CTGAGGGTCCACCAAACGTAATGCGGGGTGCATT |
| | TCGCTG_3′inverted (SEQ ID NO: 18) |
| SARS-CoV-2 N Gene | AUGUCUGAUAAUGGACCCCAAAAUCAGCGAAAUGCACC |
| Pseudovirus | CCGCAUUACGUUUGGUGGACCCUCAGAUUCAACUGGC |
| | AGUAACCAGAAUGGAGAACGCAGUGGGGCGCGAUCAA |
| | AACAACGUCGGCCCCAAGGUUUACCCAAUAAUACUGCG |
| | UCUUGGUUCACCGCUCUCACUCAACAUGGCAAGGAAG |
| | ACCUUAAAUUCCCUCGAGGACAAGGCGUUCCAAUUAAC |
| | ACCAAUAGCAGUCCAGAUGACCAAAUUGGCUACUACCG |
| | AAGAGCUACCAGACGAAUUCGUGGUGGUGACGGUAAA |
| | AUGAAAGAUCUCAGUCCAAGAUGGUAUUUCUACUACC |
| | UAGGAACUGGGCCAGAAGCUGGACUUCCCUAUGGUGC |
| | UAACAAAGACGGCAUCAUAUGGGUUGCAACUGAUGGG |
| | AGCCUUGAAUACACCAAAAGAUCACAUUGGCACCCGCA |
| | AUCCUGCUAACAAUGCUGCAAUCGUGCUACAACUUCCU |
| | CAAGGAACAACAUUGCCAAAAGGCUUCUACGCAGAAGG |
| | GAGCAGAGGCGGCAGUCAAGCCUCUUCUCGUUCCUCA |
| | UCACGUAGUCGCAACAGUUCAAGAAAUUCAACUCCAGG |
| | CAGCAGUAGGGGAACUUCUCCUGCUAGAAUGGCUGGC |
| | AAUGGCGGUGAUGCUGCUCUUGCUUUGCUGCUGCUU |
| | GACAGAUUGAACCAGCUUGAGAGCAAAAUGUCUGGUA |
| | AAGGCCAACAACAACAAGGCCAAACUGUCACUAAGAAA |
| | UCUGCUGCUGAGGCUUCUAAGAAGCCUCGGCAAAAAC |
| | GUACUGCCACUAAAGCAUACAAUGUAACACAAGCUUUC |
| | GGCAGACGUGGUCCAGAACAAACCCAAGGAAAUUUUG |
| | GGGACCAGGAACUAAUCAGACAAGGAACUGAUUACAA |
| | ACAUUGGCCGCAAAUUGCACAAUUUGCCCCCAGCGCUU |
| | CAGCGUUCUUCGGAAUGUCGCGCAUUGGCAUGGAAGU |
| | CACACCUUCGGGAACGUGGUUGACCUACACAGGUGCCA |
| | UCAAAUUGGAUGACAAAGAUCCAAAUUUCAAAGAUCA |
| | AGUCAUUUUGCUGAAUAAGCAUAUUGACGCAUACAAA |
| | ACAUUCCCACCAACAGAGCCUAAAAAGGACAAAAAGAA |
| | GAAGGCUGAUGAAACUCAAGCCUUACCGCAGAGACAGA |
| | AGAAACAGCAAACUGUGACUCUUCUUCCUGCUGCAGA |
| | UUUGGAUGAUUUCUCCAAACAAUUGCAACAAUCCAUG |
| | AGCAGUGCUGACUCAACUCAGGCCUAA (SEQ ID NO: |
| | 19) |
| 202T N gene forward primer | GACCCCAAAATCAGCGAAAT (SEQ ID NO: 20) |
| 202T N gene reverse primer | TCTGGTTACTGCCAGTTGAATCTG (SEQ ID NO: 21) |
| N gene TaqMan probe | FAM_ACCCCGCATTACGTTTGGTGGACC_BHQT (SEQ ID |
| | NO: 22) |

### Preparation of Simulated Saliva Samples

The SARS-CoV-2 pseudovirus were obtained from Beyotime Biotechnology (Shanghai, China). To mimic clinical RNA samples, the artificial saliva from Scientific Phygene (Fuzhou, China) were spiked with known concentrations of SARS-CoV-2 pseudovirus and human genomic DNA (7.89 ng/μL, Sigma Aldrich). For RNA extraction, the TIANamp Virus RNA kit from TIANGEN (Beijing, China) was applied to extract all the simulated samples according to the manufacturer's instructions.

### ID-Primers, PCR Primers and Probes Design

For the ID-Primers, we predicted the ID sequences with rationally designed Tₘ values by the NUPACK. The PCR primers and detection probes were designed using MATLAB to calculate the thermodynamic parameters. The PCR forward primer for SARS-CoV-2 pseudovirus is 2019-nCoV_N1 Forward Primer (CDC-approved). For the quencher probe, an inverted dT was added to the 3′ end to prevent DNA polymerase extension. We also screened all these sequences in BLAST analysis tool to prevent undesirable binding and utilized NUPACK to avoid secondary structures.

### Reverse Transcription

A 20 μL reverse transcription reaction was carried out by adding 1×PrimeScript buffer and 200 U PrimeScript Reverse Transcriptase (Takarabio), 13 μL template, 20 nM ID-Primer, 20 U RNase Inhibitor (Human Placenta, New England Biolabs) and 0.5 μL dNTP Mixture (10 mM, New England Biolabs) at 42° C. for 15 min and terminated at 90° C. for 5 min. Next, 40 U of Thermolabile Exonuclease I (New England Biolabs) and 2.5 μL 10×NEBuffer 3.1 (New England Biolabs) were incubated with the mixtures in a total volume 25 μL at 37° C. for 4 min and inactivated at 80° C. for 1 min.

### PCR Amplification and Melting Curve Analysis of Pooled Samples

For our method, each 25 μL PCR reaction contained 2.5 μL of the pooled samples, 10 μL Premix Ex Taq (Takarabio), 80 nM of each fluorophore probe, 80 nM quencher probe, 800 nM PCR universal forward primer, 40 nM of each reverse primer (80 nM for ID-Primer 4). The PCR program of asymmetric amplification was set as: 95° C. for 30 s and 45 cycles of 95° C. for 5 s, 64° C. for 30 s. Melting curve analysis was conducted with a program that begins with a denaturation step of 1 min at 95° C., a hybridization step of 5 min at 35° C. and followed by stepwise temperature increase from 35° C. to 80° C. with 0.5° C. increment per step for fluorescence measurement in the FAM and HEX channels. For Dorfman-based pooling test, each 20 μL PCR mix included 2 μL pooled samples, 200 nM each of PCR forward and reverse primers, 400 nM TaqMan probes and 10.8 μL One Step PrimeScript RT-PCR mixture (Takarabio). The corresponding PCR protocol was set as 42° C. for 10 min, 95° C. for 30 s and 45 cycles of 95° C. for 5 s, 60° C. for 30 s. All the PCR reactions were performed on the CFX Opus 96 Real-Time PCR System (Bio-Rad).

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1—Working Principle

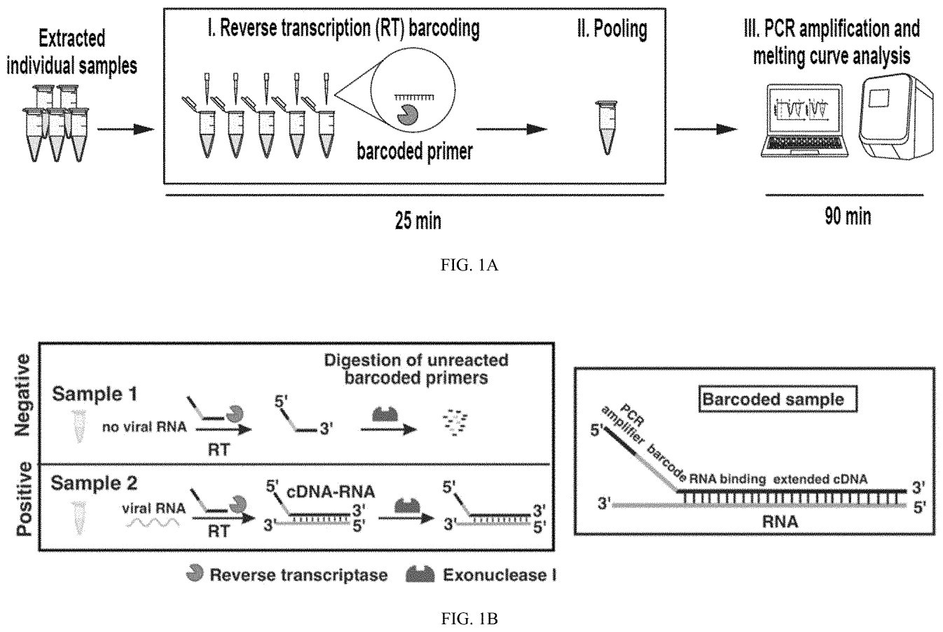

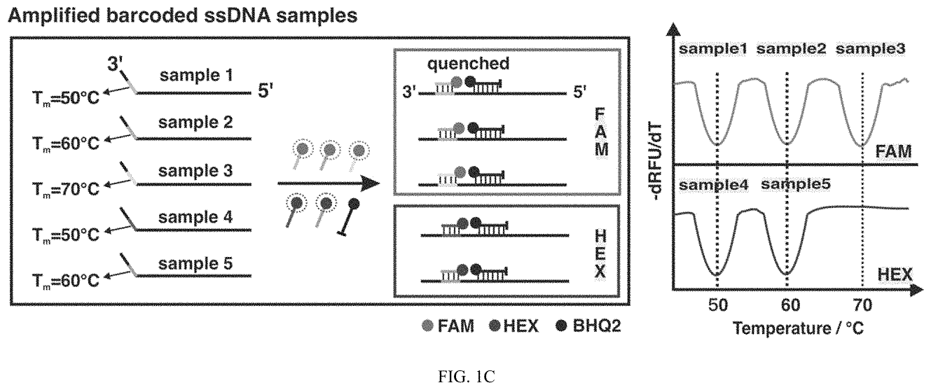

The workflow of Uni-Pool includes reverse transcription (RT), sample pooling, PCR amplification and melting curve analysis (FIG. 1A). First, the extracted samples are tagged with a unique ID-Primer through RT. The sequence of ID-Primer con-sists of three parts (FIG. 1B): the target-specific region complementary to the RNA target of interest, the ID region that provides a unique identifier sequence differentiated by its Tₘ (by altering the sequence), and the amplifier sequence for the subsequent PCR primer binding region. The sample-specific ‘ID-Primer’ is added to each sample specifically. If the sample contains the viral RNA, as illustrated in the sample 2 of FIG. 1B, the ID-Primer acts as the RT primer, thus generating a cDNA-RNA duplex upon the addition of reverse transcriptase without Ribonuclease H (RNase H) activity. On the contrary, the primers remain as single-stranded DNA in the absence of the RNA target (as illustrated in sample 1). Then, exonuclease I is added to each sample. Since it can only degrade single stranded DNA from 3′ to 5′ end, it selectively digests all the un-used primers and only the cDNA-RNA duplexes remain intact. Thus, each positive sample with the viral RNA is labelled with a special ID sequence that can be differentiated in the next step. The ID-Samples are then pooled together for downstream analysis. In case of PCR melting curve analysis of the pooled samples as shown in FIG. 1C, the grouped samples are amplified via asymmetric PCR to generate single-stranded amplicons from the tagged cDNA-RNA targets in the pool. The melting curve probe set comprises of a probe with the fluorophore (complementary to the ID region, called F probe) and a probe with the quencher (complementary to the cDNA region adjacent to the F probe-binding region, called Q probe). The Q probe is designed to be long enough to make sure that its Tₘ value is higher than all the F probes, thus the Q probe will remain anchored to the tar-get strand and only F probe will be responsible for any signal changes. During the melting curve analysis, the F and Q probes will hybridize to their corresponding sites at 45° C., and at this point, the fluorescence signal is quenched. When the temperature reaches the Tₘ value of the F probe, the F probe will be released and would yield a sharp increase in fluorescence signal. By designing F-probes of different Tₘ values, the first derivative −dRFU/dT peak of the melting curve analysis can be correlated to the presence of its corresponding positive sample. Moreover, the amount of samples pooled in the Uni-Pool system could be further expanded by a “2D” design using F probes labelled by different fluorophores.

### Example 2—Sample-Specific Detection of Pooled Samples in One Fluorescence Channel

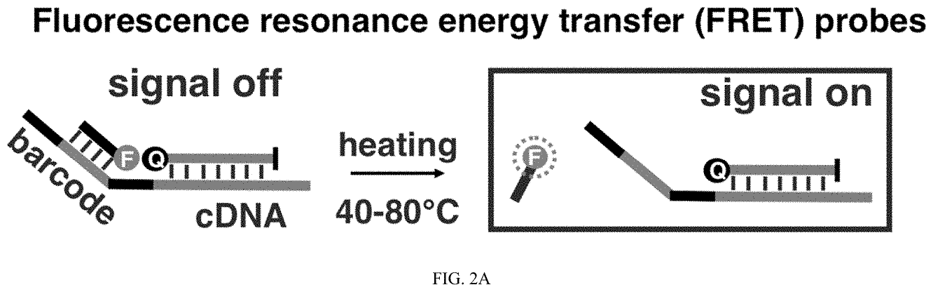

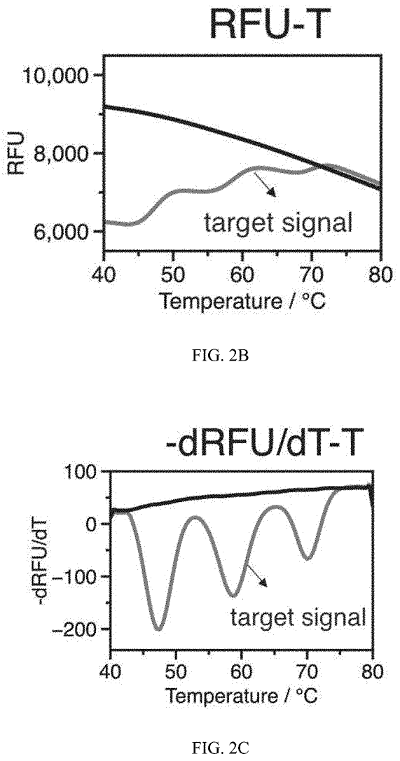

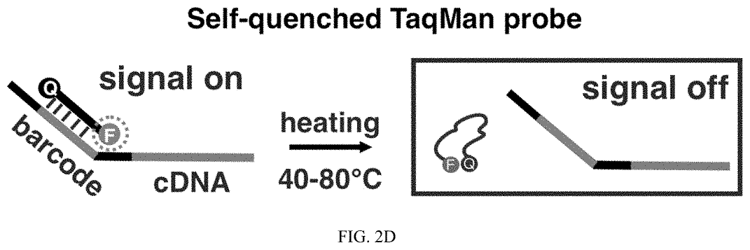

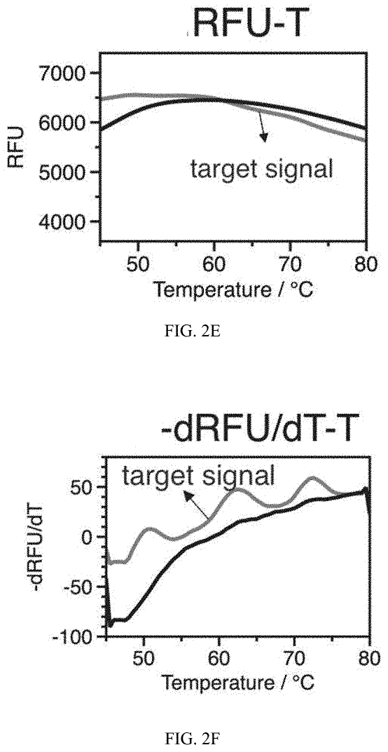

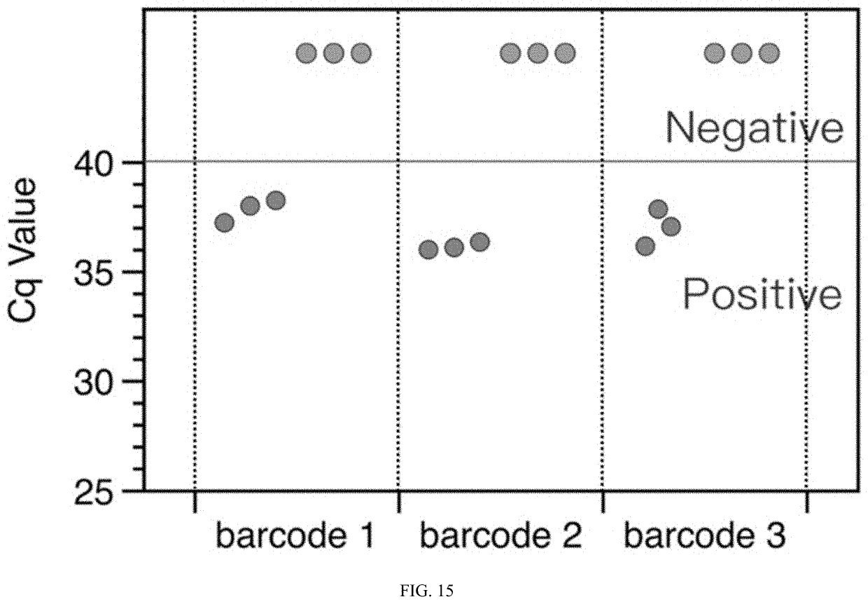

Our assay was first tested for its robustness in the FAM channel. Three samples were labelled with three different ID-Primers, each with its corresponding F probe. Three F probes were covalently modified with FAM and designed to have Tₘ values of 10° C. difference among each other. Specifically, Tₘ values of 50° C., 60° C., and 70° C. in FAM channel corresponded to sample 1, sample 2 and sample 3, respectively. To test the feasibility of adding ID-Primers to each target sequence using RT, an individual sample was analyzed by RNA-specific qPCR separately. As shown in FIG. 15, samples with viral RNA could be differentiated from the samples without RNA, suggesting the success of adding ID-Primers to each sample using RT. Next, in the multiplexed melting curve analysis, we utilized two different designs of fluorescent and quencher probe pairs: one with two separate fluorescent probe (F-probe) and quencher probe (Q-probe) (i.e., fluorescence resonance energy transfer (FRET) probes) and the other with one single strand probe labelled with quencher and reporter dyes (e.g., self-quenched TaqMan probe). As shown in FIGS. 2A-2F, the signal-on FRET probes approach employing separate F and Q probes shows a sharper melt peak, after taking the first derivative of the fluorescence vs. temperature chart (−dRFU/dT), than that of the signal-off approach with the self-quenched TaqMan probe. Although both designs were able to identify the three RNA-contained samples in a pool, the first derivative of the melting curve chart using the self-quenched TaqMan probe showed an increasing background signal in no template control (NTC), preventing a clear differentiation from those of the positive samples (FIG. 2F). Nevertheless, the FRET probe design displayed a steady background −dRFU/dT curve in NTC, easily distinguishable with the positive samples (FIG. 2C). Moreover, the false positive results may arise without complete removal of unreacted ID-Primers by exonuclease I in the TaqMan probe-based strategy, as observed in a multiplexed NGS assay utilizing a similar strategy²⁶. On the other hand, the FRET probe-based strategy was designed to have the Q probe targeting the cDNA region of the amplicon to avoid the false negative results caused by the unreacted ID-Primers. Hence, the FRET probe-based strategy producing much sharpened signal from that of the background (FIG. 2C) will be used in our Uni-Pool system throughout the following studies.

To check any cross interaction among negative and positive samples in the same pool that may lead to false positive/negative results, we characterized possible combinations of three samples (all positive cases, all negative cases, one positive case in three, two positive cases in three) in one pool. Samples of different scenarios were identified in FIGS. 2G-2K.

### Example 3—Analytical Performance of 2D Melting Curve System

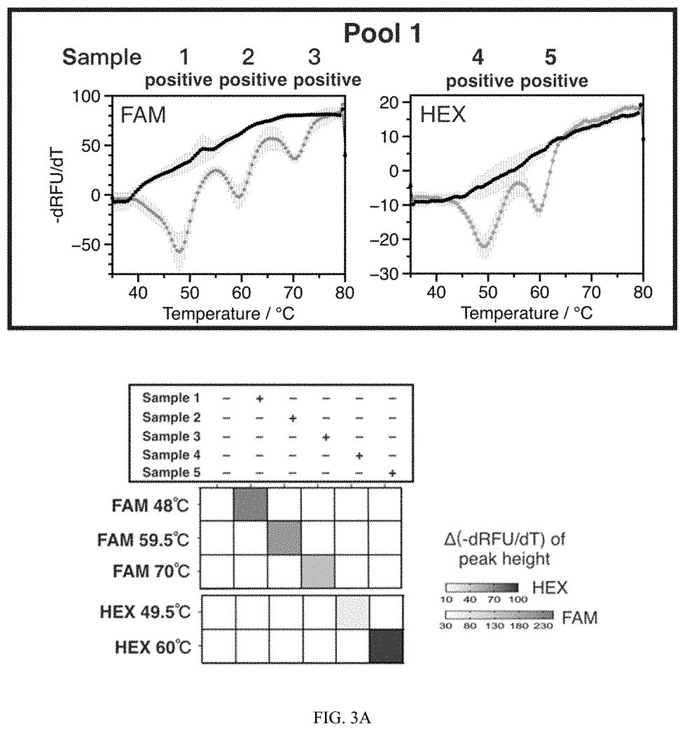

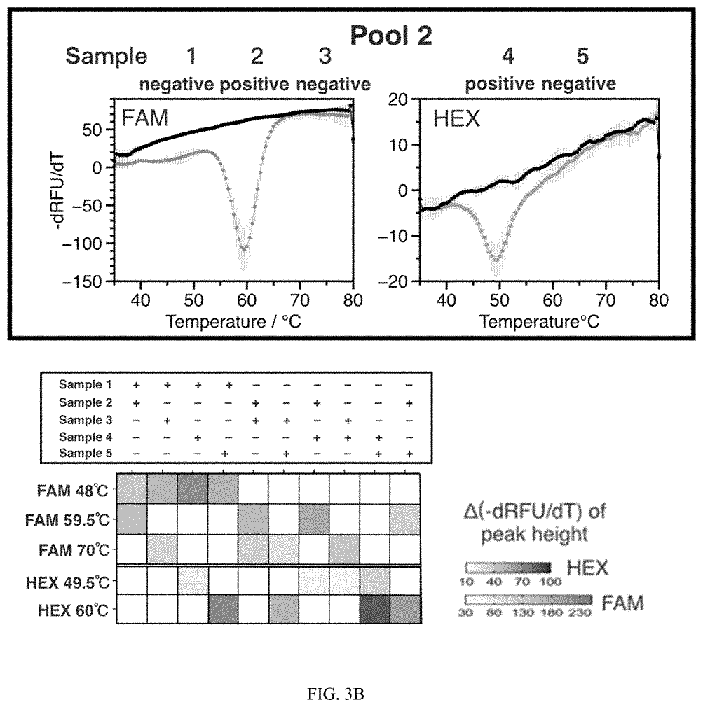

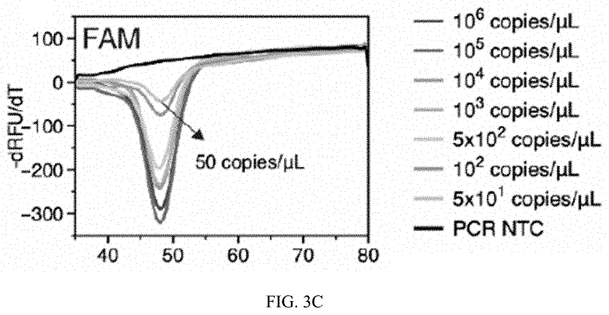

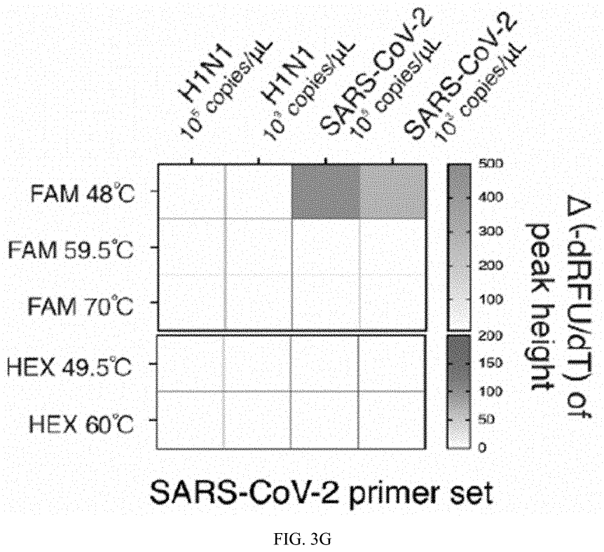

One advantage of the multiplex melting curve analysis is that the test capacity can be expanded by employing multiplex detection probes with different Tₘ value and in different fluorescent channels (2D). In the Uni-Pool assay, five samples were labelled with five different ID-Primers with their corresponding F probe. Three of the F probes were covalently modified with FAM with Tₘ values of 50° C., 60° C., and 70° C., respectively. The other two F probes were covalently modified with HEX fluorophore with Tₘ values of 50° C. and 60° C., respectively. The identification of each target in the pool was revealed by the combination of different fluorophore and Tₘ values of the probes of the corresponding amplicon. As a result, 2 to the power of 5 combinations for five individual samples in one pool can all be distinguished (FIGS. 3A-3B) by the Uni-Pool.

After verifying feasibility of this 2D multiplex melting curve analysis in the Uni-Pool, the sensitivity and specificity of this Uni-Pool assay was evaluated by detecting the serially diluted RNA samples ranging from 5×10¹ to 10⁶ copies/μL (FIGS. 3C-3G). The lowest detectable concentration was 5×10¹ copies/μL in a pool with only one positive case in FAM or HEX channel displaying a discernible melting peak against that of the NTC. To evaluate the influence of concentration variation on the overlapping of two adjacent positive melting peaks, we performed the sensitivity test on the 5-sample pools containing two positive samples corresponding to two adjacent melting peaks and three negative samples. When the concentration of one of the two positive samples was fixed at 10⁶ copies/μL, the lowest detectable concentration of the other positive sample was 5×10¹ copies/μL in FAM channel and 10² copies/μL in HEX channel (FIGS. 3E-3F). This minimal detectable concentration in HEX was higher than that observed in the pools of single positive sample. This is possibly because the high concentration samples may outcompete other lower concentration samples in the pool in the competition for amplification primers and detection probes within one reaction. Indeed, Uni-Pool strategy displayed a sensitivity lower than that of commercial RT-PCR kits²⁷. This is probably caused by the dilution effect after the separate RT step and the competition inhibition effect among the pooled samples. Notably, a previous study suggested that in case of large-scale screening where efficiency of testing is highlighted, a detection limit of 10² copies/μL would be sufficient²⁸. Next, the specificity of our Uni-Pool assay for SARS-CoV-2 was tested by examining its cross-reactivity against Influenza H1N1 RNA with different concentrations, and no non-specific responses were observed (FIG. 3G). The results show that there was no cross-reaction of this system with other respiratory pathogen and indicate the good specificity of this assay.

### Example 4—Detection of Simulated Saliva Samples

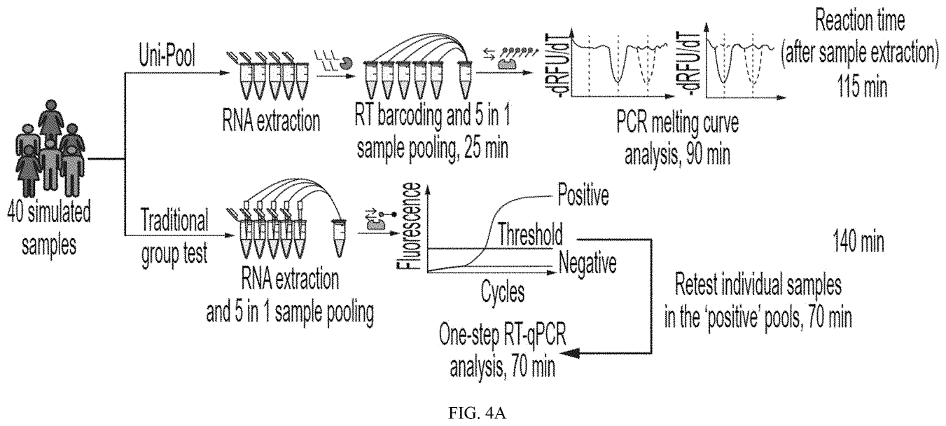

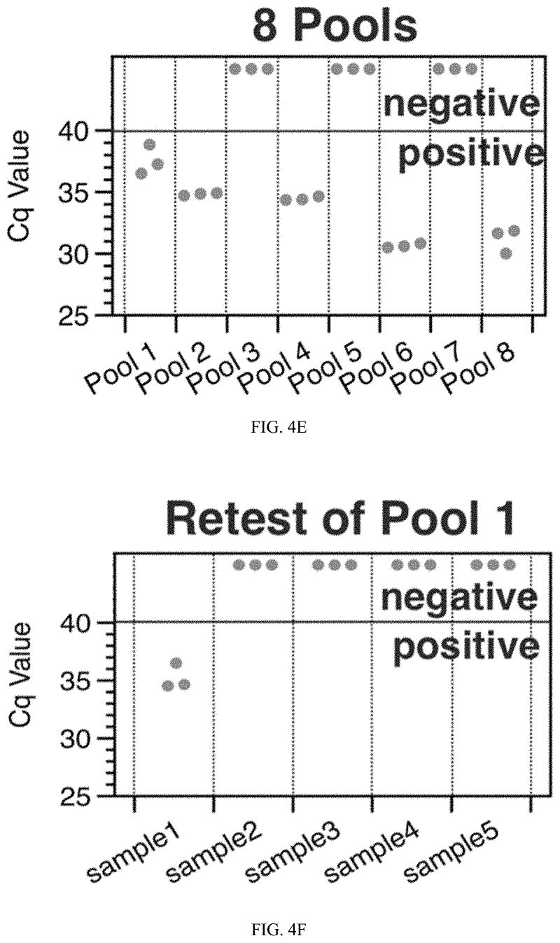

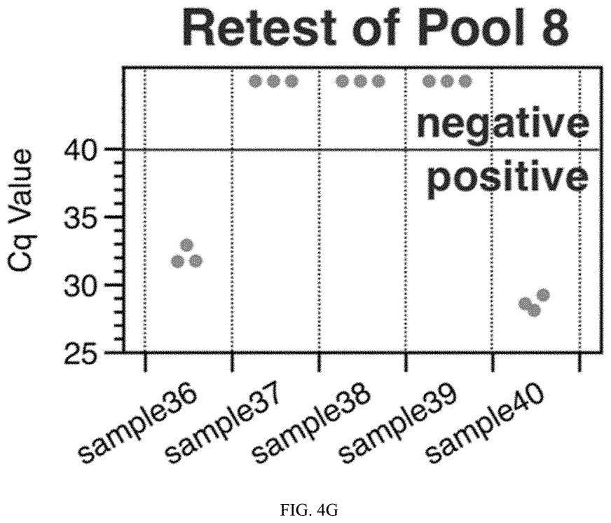

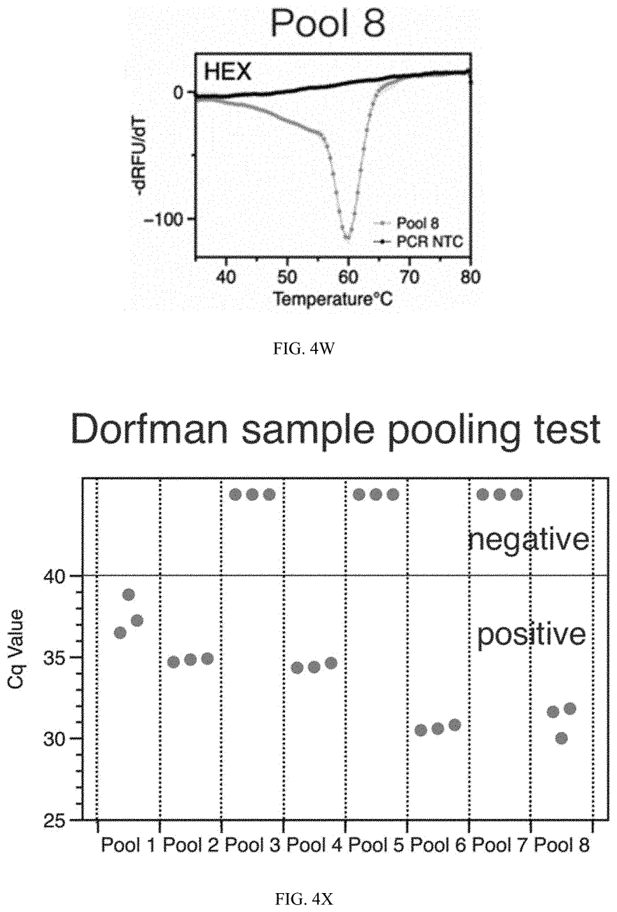

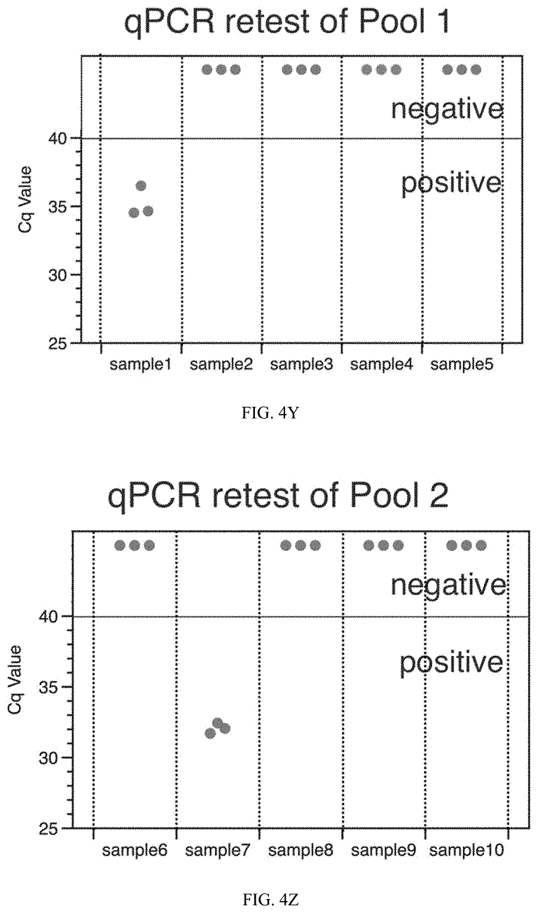

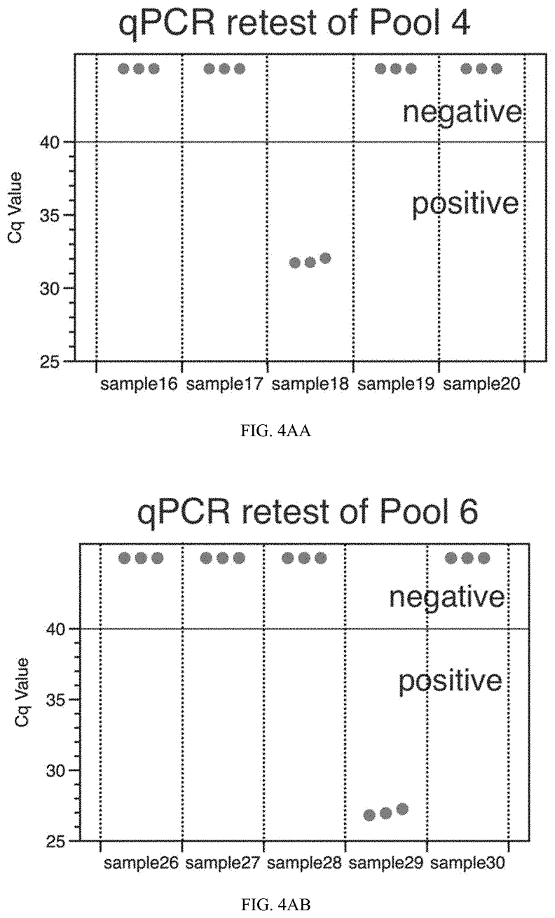

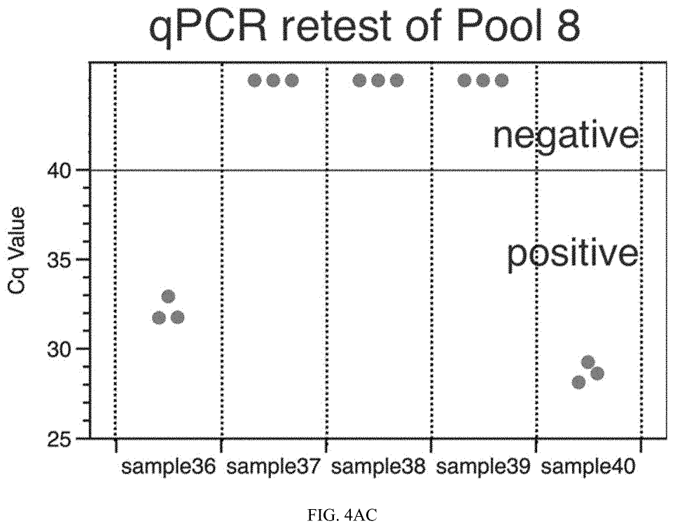

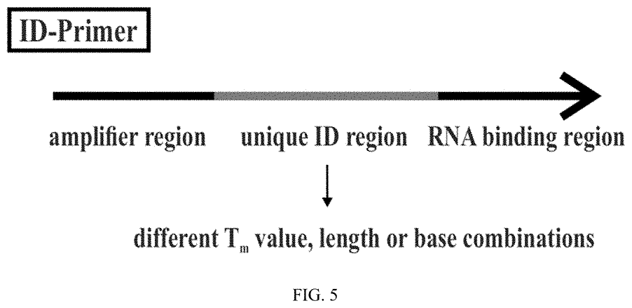

To investigate the feasibility of Uni-Pool assay in the clinical settings, we tested the simulated saliva samples using our Uni-Pool in comparison with conventional group test simultaneously. We prepared 40 simulated saliva samples. 6 of them were spiked with the SARS-CoV-2 pseudo virus of different loads (min: 10 copies/μL, max: 103 copies/μL) and the remaining 34 samples were all negative so that the prevalence rate in this test was determined to be 15%. These 40 samples were divided into 8 pools randomly and each pool contained five individual samples. The turnaround time of the Uni-Pool assay to identify the 6 positive samples from the 40-sample group was 115 min which was shorter than the 140 min taken by the conventional group test (FIG. 4A). The conventional group testing was not efficient for samples with the prevalence rate as high as 15% in our experiment, which is consistent with the previous studies on Dorfman sample pooling strategy²⁹. On the other hand, the Uni-Pool assay only required 8 pooled PCR reactions along with the following melting curve analysis in this experiment, significantly reducing the cost of reagent and human resources compared with the 78 RT-qPCR reactions used by the secondary test in conventional group test. As seen in FIGS. 4B-4G, all positive specimens with viral loads ranged from 10 to 103 copies/μL were sample-specifically identified from the negative specimens within one round of pooled reaction. Notably, the sensitivity of detecting SARS-CoV-2 pseudo virus (10 copies/μL) using Uni-Pool assay was found to be higher than that of detecting synthetic RNA samples (50 copies/μL), which can be attributed to the loss of RNA targets on repeated freezing and thawing during transportation. Additionally, the results of our sample-specific pooled testing were in good agreement with the qPCR results as shown in FIGS. 4B-4I. This indicates that our method can achieve efficient, accurate and sensitive detection of pooled samples irrespective of the prevalence of the infectious agent.

### Example 5—Detection of Pooled RNA Samples by Multiplex Melting Curve System

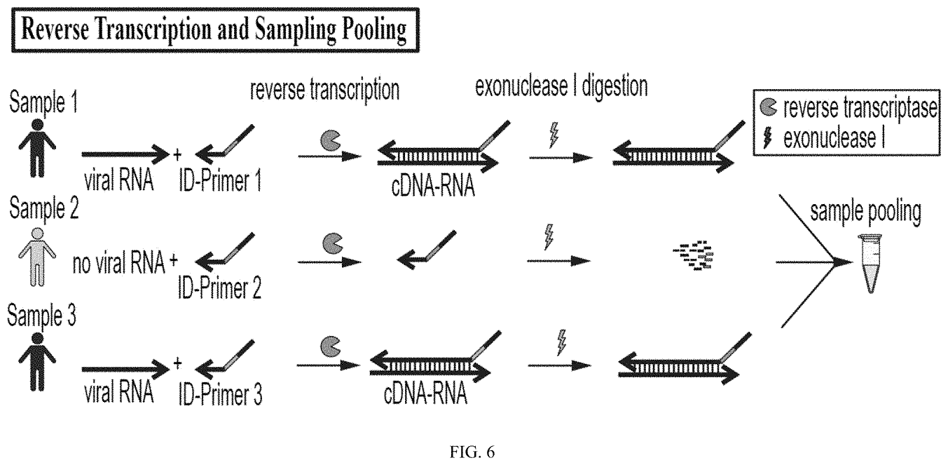

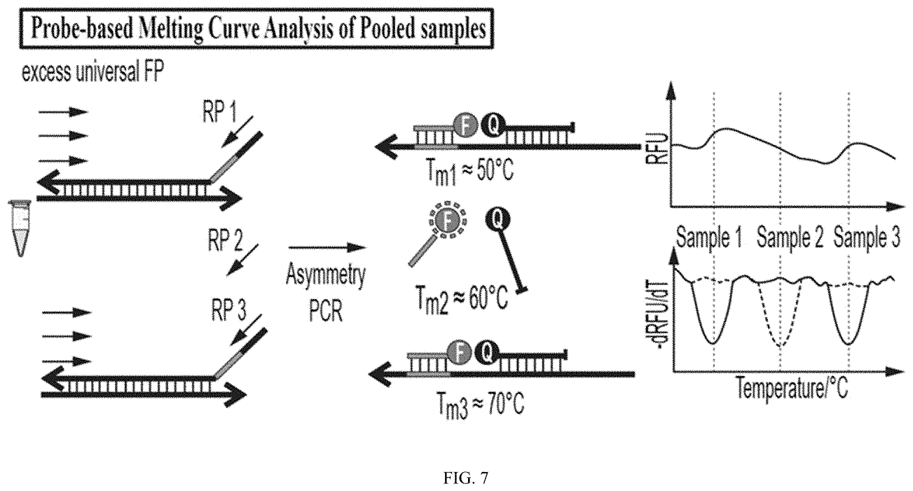

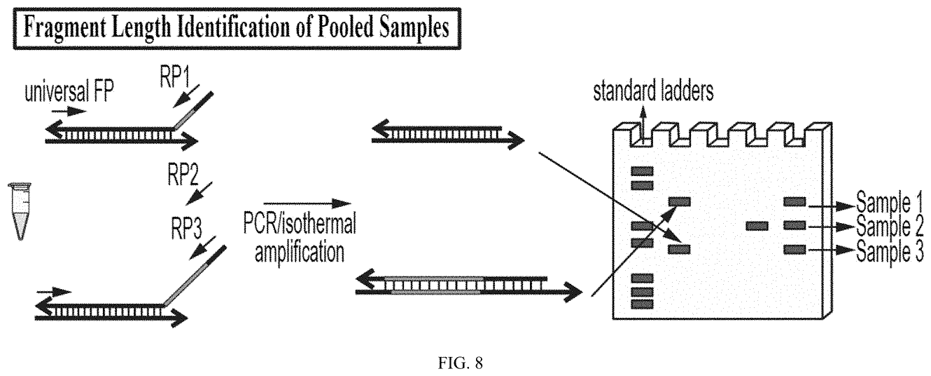

Three samples were designed to be labelled with three different ID-Primers each with its own corresponding fluorophore-containing hybridization probe or F-probe through reverse transcription (FIG. 6). The ID regions of three ID-Primers were designed to have Tₘ values of 10° C. difference among each other. Three of the F probes were covalently modified with FAM, and designed to have Tₘ's of approximately 50° C., 60° C., and 70° C. Next, the ID-Samples will be pooled together and then directly used for the one-pot multiplex melting curve analysis. In the pool, the tagged cDNA-RNA templates will generate abundant complementary single strand amplicons through asymmetric PCR with abundant forward primer. Next, the matching fluorophore and quencher probe sets hybridize to the homologous amplicons at 45° C. By increasing the reaction temperature, compared to the unhybridized fluorophore probes, the first derivative of the fluorescent intensity vs. temperature (−dRFU/dT) is an indication of positive (with peak) or negative (without peak) of the screening outcome (FIG. 7). Meanwhile, the test capacity can be further expanded by employing multiplex detection probes with different Tₘ values and in different fluorescent channels. For example, six individual samples can be labelled with six different ID-Primers with six F probes. Three of the F probes will be modified with FAM with Tₘ value of 50° C., 60° C., and 70° C., respectively. The other three F probes will be labelled with HEX fluorophore with Tₘ value of 50° C., 60° C., and 70° C., respectively.

### Example 6—Detection of Pooled RNA Samples Using Fragment Length Identification

In this case, we designed three ID sequences of different lengths (10 nts, 15 nts, 20 nts) appended to the 5′end of three ID-Primers that were specific to the target RNA of interest (FIG. 8). Each of these ID-Primers was added to an individual sample after RNA extraction, which will generate an RNA/DNA duplex upon the addition of reverse transcriptase only if the sample contains the virus. Then, the samples are pooled together, and the amplification primers will be added to generate a double-stranded product of a unique length that can be traced to the length of the ID-Primers initially added. After symmetric PCR amplification or isothermal based amplification of the pooled samples, the products can then be separated and analyzed by the gel electrophoresis. Based on the size selection principle, each sample in the pools was recognized according to the bands appeared in the expected region of gel. If no signal is detected, then all individual samples are declared negative, but if a signal is present, the length of the product will reveal which sample is positive (FIG. 8). The detection volume can also be enlarged by designing multiple ID-Primers with different length and by enhancing the separation resolution of the size-separation technique.

### Example 7—Real Time Detection of RNA Pooled Samples Using RT-QPCR Analysis by Taqman Hydrolysis Probes

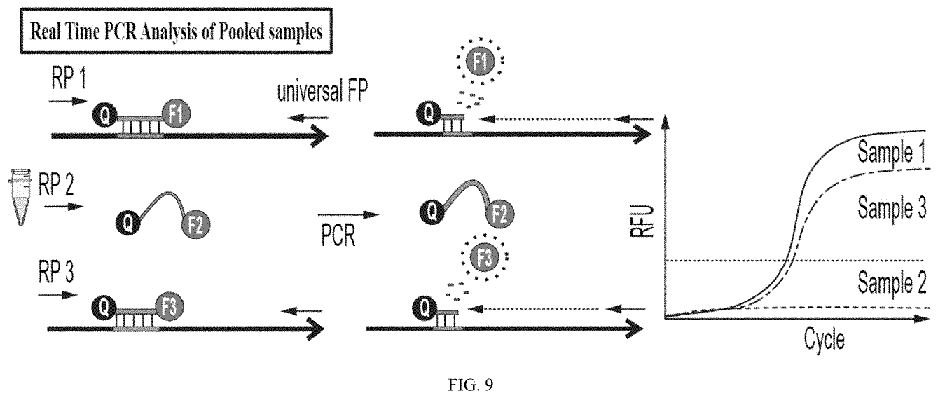

The qualitative and quantitative analysis of pooled samples can be achieved by the introduction of TaqMan hydrolysis probes, which are dual labelled with different fluorophores and its corresponding quenchers. Different samples were labelled with different ID-Primers with different base compositions in the ID region and the ID region is fully complementary to its relative dual labelled TaqMan hydrolysis probes. As shown in FIG. 9, during the real-time qPCR, the TaqMan probes are hybridized with its complementary targets and then be hydrolysed by the exonuclease activity of the polymerase to exhibit the amplification plots. Therefore, according to the cycle threshold value of these amplification curves, we can determine which samples in the pool are positive or negative and how many copies of viral RNA are present in the positive samples, thereby making judgments on the severity of the patients' infection. In addition, the detection capacity depends on the numbers of the fluorescence detection channels on the PCR thermocycler.

### Example 8—Detection of RNA Pooled Samples with an Electrochemical Readout

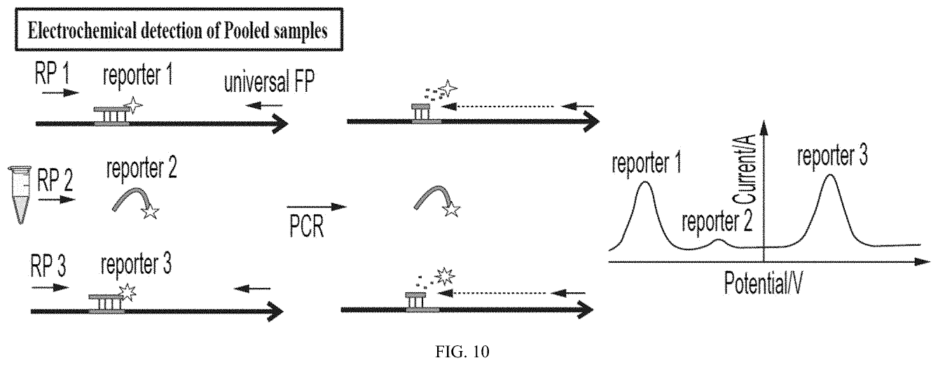

A modification of the previous example can be introduced by using the detection probes that labeled with electroactive reporters with non-overlapping redox potentials, i.e., ferrocene, methylene blue, anthraquinone. The multichannel electrochemical workstation can obtain the end-point signal readout for the amplified pooled samples. As only those samples with the RNA virus will have abundant amplicons generated, the redox peak at certain potential from a specific electroactive reporter corresponds to the positive sample (FIG. 10).

### Example 9—Patient-Specific Pooling Strategy for Multiple RNA Samples by Magnetic Separation

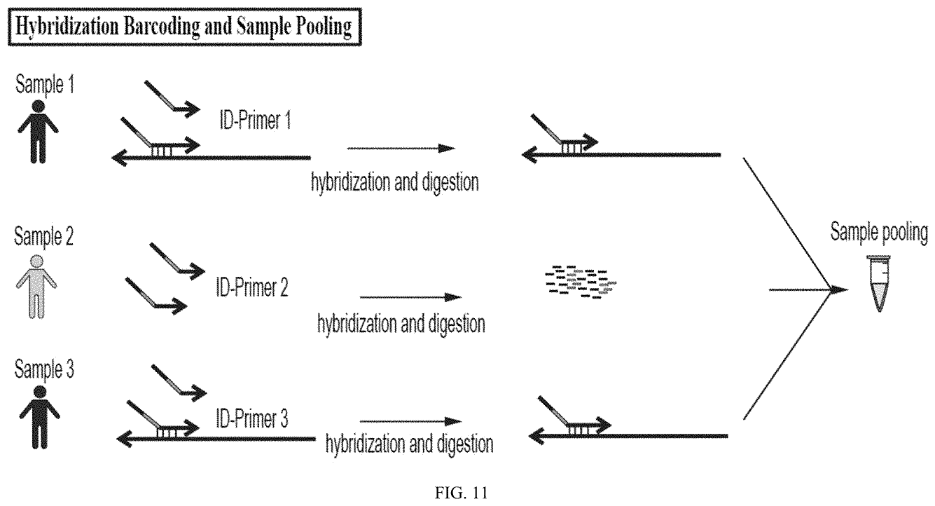

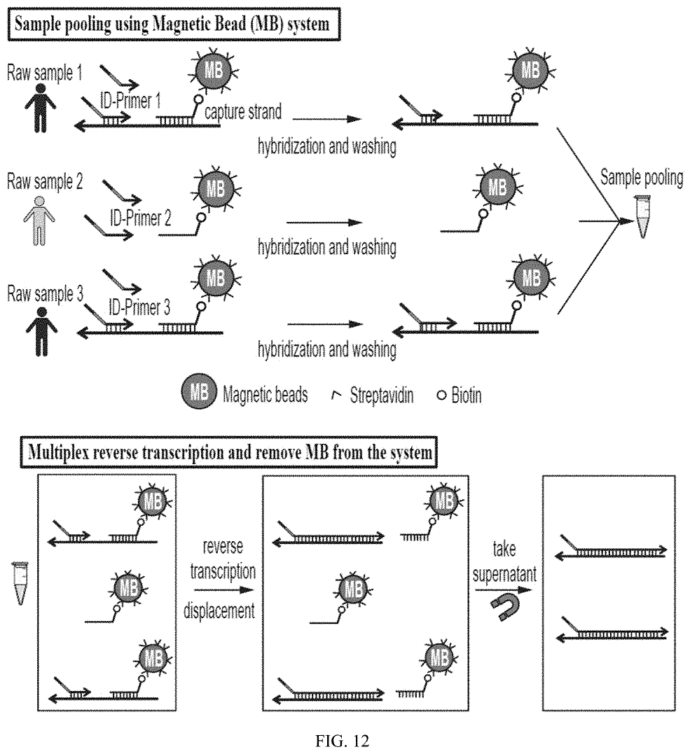

A fifth case is presented in this invention where the RNA extraction and digestion of unreacted ID-Primers by enzyme (FIG. 11) are not need. A biotin modified RNA capture strand is attached to a streptavidin-modified magnetic bead (MB) via the streptavidin-biotin interaction (FIG. 12). The lysis buffer, RNA capture strand with MB and the ID-Primers are added to the individual raw specimen firstly. After incubation at room temperature for 10 mins, the samples will be hybridized with the MB with RNA capture strand and the ID-Primers and then the unreacted ID-Primers can be washed away by the washing buffer. At this stage, the labelled individual samples will be grouped together for multiplexed reverse transcription, amplification, and detection. Considering the MB will influence the signal readout, another alternative is to remove the MB with capture strand by the displacement ability of reverse transcriptase. Because the RNA capture strand can be designed to the right (3′end) of the ID-Primers, which will act as the reverse transcription primers, thereby after multiplexed reverse transcription and magnetic separation, the MB with capture strand will be separated from this system without any additional complicated steps (FIG. 12).

### Example 10—Detection of RNA Pooled Samples by Melting Curve System Using a Universal Probe Set

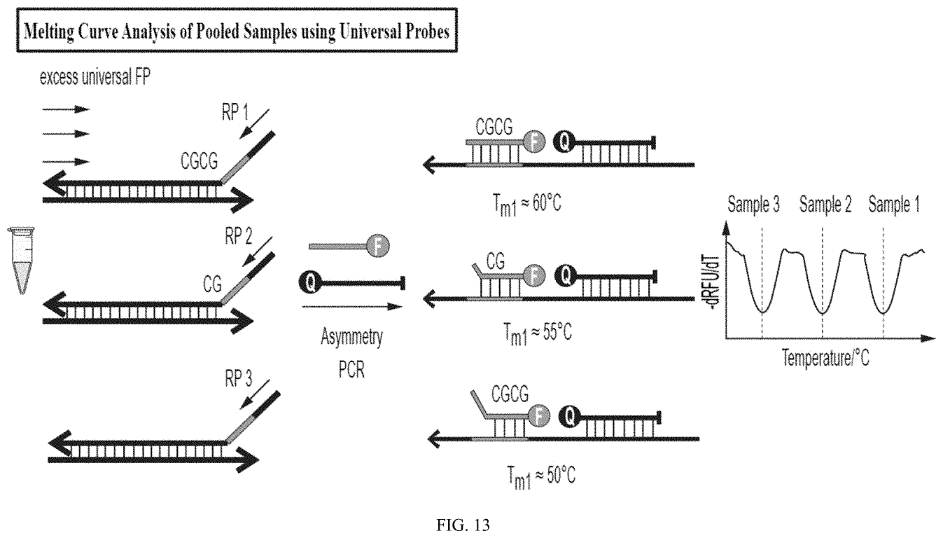

A universal probe set can be involved to further decrease the complexity of the melting curve detection system. Here, this universal probe set consists of two probes (FIG. 13), one is labelled with fluorophore (F-probe), and another is labelled with quencher (Q-probe). The major difference of this design compared to the previous design (FIG. 7) is that this F-probe can detect three ID sequences at the same time. If there have three ID positive samples (sample 1, sample 2 and sample 3) in the pool, the F-probe is designed to be fully complementary to the ID region of sample 1. For the sample 1, it has 2 nucleotides (CG) more than the sample 2 in the ID region, and 4 nucleotides (CGCG) more than the sample 3. These 2 or 4 nucleotides difference can lead to the ID sequence has different Tₘ value during the melting curve analysis, because when F probe hybridizes with the sample 2, there will form a 2 nts dangle end and for the sample is a 4 nts dangle end. This design can yield a higher throughput and efficient detection of multiple targets simultaneously.

### Example 11—Detection of Pooled Samples Using Close-Quenched Melting Curve Probe Sets

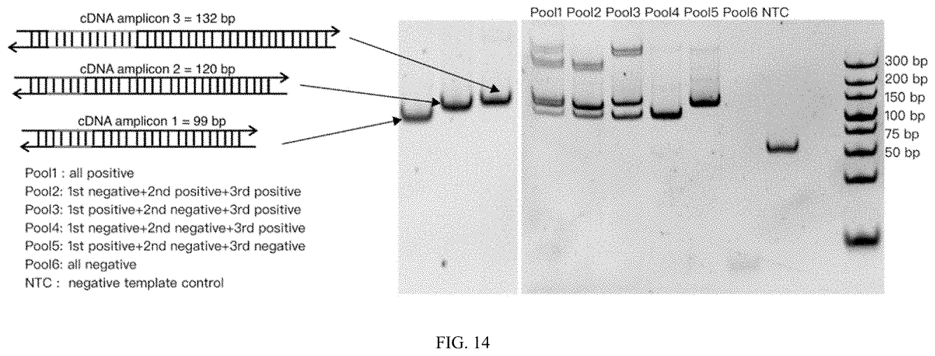

Five samples were designed to be labeled with five different ID-Primers each with its own corresponding fluorophore-containing hybridization probe or F-probe. Three of the F probes were covalently modified with FAM, and designed to have Tₘ's of approximately 50° C., 60° C., and 70° C., while the other two F probes, were covalently modified with HEX fluorophore, and have Tₘ's of 50° C. and 60° C. The concentration of the synthetic SARS-CoV-2 positive template used is 103 copies/μl. Only the sharp downward peak in the designed Tₘ can be regarded as the evidence for the existence of the positive sample in the pool. The total 32 possible combinations of pools for five samples can all be identified even under different prevalence (FIGS. 14A-14G). As for the sensitivity test, the melting curve results of the pools with one or two positive samples show that the lowest concentration that can be detected is 50 copies/μl in FAM channel and 100 copies/μl in HEX channel (FIGS. 15A-15D).

### Example 12—Detection of Pooled Samples Using Fragment Length Identification

Three individual samples were labeled with three ID-Primers with different lengths (39 nts, 60 nts, 72 nts) through reverse transcription. Then the three ID-Samples were pooled together. After symmetric PCR amplification of the pooled sample, when three samples were all positive in one pool, the amplicons with relative lengths (99 bp, 120 bp, 132 bp) were generated and separated by the gel electrophoresis. Based on the size selection principle, the pools with different positive rates were recognized according to the bands appeared in the expected region of gel. The 12 nts difference of tag primers was shown to be sufficient to distinguish two samples in the same pools (FIG. 16).

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

(1) Broughton, J. P.; Deng, X.; Yu, G.; Fasching, C. L.; Servellita, V.; Singh, J.; Miao, X.; Streithorst, J. A.; Granados, A.; Sotomayor-Gonzalez, A.; Zorn, K.; Gopez, A.; Hsu, E.; Gu, W.; Miller, S.; Pan, C. Y.; Guevara, H.; Wadford, D. A.; Chen, J. S.; Chiu, C. Y. CRISPR-Cas12-Based Detection of SARS-CoV-2. Nat. Biotechnol. 2020, 38 (7), 870-874.
(2) Yan, Y.; Chang, L.; Wang, L. Laboratory Testing of SARS-CoV, MERS-CoV, and SARS-CoV-2 (2019-NCoV): Current Status, Challenges, and Countermeasures. Rev. Med. Virol. 2020, 30 (3), 1-14.
(3) Excler, J. L.; Saville, M.; Berkley, S.; Kim, J. H. Vaccine Development for Emerging Infectious Diseases. Nat. Med. 2021, 27 (4), 591-600.
(4) Ferretti, L.; Wymant, C.; Kendall, M.; Zhao, L.; Nurtay, A.; Abeler-Dörner, L.; Parker, M.; Bonsall, D.; Fraser, C. Quantifying SARS-CoV-2 Transmission Suggests Epidemic Control with Digital Contact Tracing. Science (80-.). 2020, 368 (6491), 0-7.
(5) Eberhardt, J. N.; Breuckmann, N. P.; Eberhardt, C. S. Multi-Stage Group Testing Improves Efficiency of Large-Scale COVID-19 Screening. J. Clin. Virol. 2020, 128 (April), 104382.
(6) Khan, P.; Aufdembrink, L. M.; Engelhart, A. E. Isothermal SARS-CoV-2 Diagnostics: Tools for Enabling Distributed Pandemic Testing as a Means of Supporting Safe Reopenings. ACS Synth. Biol. 2020, 9 (11), 2861-2880.
(7) Zhang, C.; Zheng, T.; Wang, H.; Chen, W.; Huang, X.; Liang, J.; Qiu, L.; Han, D.; Tan, W. Rapid One-Pot Detection of SARS-CoV-2 Based on a Lateral Flow Assay in Clinical Samples. Anal. Chem. 2021.
(8) Mutesa, L.; Ndishimye, P.; Butera, Y.; Souopgui, J.; Uwineza, A.; Rutayisire, R.; Ndoricimpaye, E. L.; Musoni, E.; Rujeni, N.; Nyatanyi, T.; Ntagwabira, E.; Semakula, M.; Musanabaganwa, C.; Nyamwasa, D.; Ndashimye, M.; Ujeneza, E.; Mwikarago, I. E.; Muvunyi, C. M.; Mazarati, J. B.; Nsanzimana, S.; Turok, N.; Ndifon, W. A Pooled Testing Strategy for Identifying SARS-CoV-2 at Low Prevalence. Nature 2021, 589 (7841), 276-280.
(9) Dorfman, R. The Detection of Defective Members of Large Populations Author (s): Robert Dorfman Source: The Annals of Mathematical Statistics, December, 1943, Vol. 14, No. 4 (December 1943), Pp. Published by: Institute of Mathematical Statistics Stable URL: Ht. Math. Stat. 1943, 14 (4), 436-440.
(10) Johnson, N. L.; Kotz, S.; Rodriguez, R. N. Dorfman-Sterrett Screening (Group Testing) Schemes and the Effects of Faulty Inspection. http://dx.doi.org/10.1080/03610928908829979 2007, 18 (4), 1469-1484.
(11) Van Zyl, G. U.; Preiser, W.; Potschka, S.; Lundershausen, A. T.; Haubrich, R.; Smith, D. Pooling Strategies to Reduce the Cost of HIV-1 RNA Load Monitoring in a Resource Limited Setting. Clin. Infect. Dis. 2011, 52 (2), 264-270.
(12) Singer, R. S.; Cooke, C. L.; Maddox, C. W.; Isaacson, R. E.; Wallace, R. L. Use of Pooled Samples for the Detection of Salmonella in Feces by Polymerase Chain Reaction. J. Vet. Diagnostic Investig. 2006, 18 (4), 319-325.
(13) Van, T. T.; Miller, J.; Warshauer, D. M.; Reisdorf, E.; Jernigan, D.; Humes, R.; Shulta, P. A. Pooling Nasopharyngeal/Throat Swab Specimens to Increase Testing Capacity for Influenza Viruses by PCR. J. Clin. Microbiol. 2012, 50 (3), 891-896.
(14) Verdun, C. M.; Fuchs, T.; Harar, P.; Elbrachter, D.; Fischer, D. S.; Berner, J.; Grohs, P.; Theis, F. J.; Krahmer, F. Group Testing for SARS-CoV-2 Allows for up to 10-Fold Efficiency Increase across Realistic Scenarios and Testing Strategies. medRxiv 2020.
(15) Noriega, R.; Samore, M. H. Increasing Testing Throughput and Case Detection with a Pooled-Sample Bayesian Approach in the Context of COVID-19. bioRxiv 2020.
(16) Z, Z.; RM, M.; J, G.; RE, W.; DA, M.; DP, M.; J, S.; YP, S. Pooled PCR Testing Strategy and Prevalence Estimation of Submicroscopic Infections Using Bayesian Latent Class Models in Pregnant Women Receiving Intermittent Preventive Treatment at Machinga District Hospital, Malawi, 2010. Malar. J. 2014, 13 (1).
(17) S, F.; U, K.; AK, N.; A, A. Sample Pooling for Real-Time PCR Detection and Virulence Determination of the Footrot Pathogen Dichelobacter Nodosus. Vet. Res. Commun. 2017, 41 (3), 189-193.
(18) Munoz-Zanzi, C.; Thurmond, M.; Hietala, S.; Johnson, W. Factors Affecting Sensitivity and Specificity of Pooled-Sample Testing for Diagnosis of Low Prevalence Infections. Prev. Vet. Med. 2006, 74 (4), 309-322.
(19) Xiong, W.; Lu, H.; Ding, J. Determination of Varying Group Sizes for Pooling Procedure. Comput. Math. Methods Med. 2019, 2019.
(20) Shental, N.; Levy, S.; Wuvshet, V.; Skorniakov, S.; Shalem, B.; Ottolenghi, A.; Greenshpan, Y.; Steinberg, R.; Edri, A.; Gillis, R.; Goldhirsh, M.; Moscovici, K.; Sachren, S.; Friedman, L. M.; Nesher, L.; Shemer-Avni, Y.; Porgador, A.; Hertz, T. Efficient High-Throughput SARS-CoV-2 Testing to Detect Asymptomatic Carriers. Sci. Adv. 2020, 5961, eabc5961.
(21) Schmid-burgk, J. L.; Li, D.; Feldman, D.; Strecker, J.; Cleary, B.; Regev, A. LAMP-Seq: Population-Scale COVID-19 Diagnostics Using a Compressed Barcode Space. bioRxiv 2020.
(22) Chappleboim, A.; Joseph-Strauss, D.; Rahat, A.; Sharkia, I.; Adam, M.; Kitsberg, D.; Fialkoff, G.; Lotem, M.; Gershon, O.; Schmidtner, A. K.; Oiknine-Djian, E.; Klochendler, A.; Sadeh, R.; Dor, Y.; Wolf, D.; Habib, N.; Friedman, N. ApharSeq: An Extraction-Free Early-Pooling Protocol for Massively Multiplexed SARS-CoV-2 Detection. medRxiv 2020, 33.
(23) Hossain, A.; Reis, A. C.; Rahman, S.; Salis, H. M. A Massively Parallel COVID-19 Diagnostic Assay for Simultaneous Testing of 19200 Patient Samples. Google Docs, March 2020.
(24) Huang, Q.; Liu, Z.; Liao, Y.; Chen, X.; Zhang, Y.; Li, Q. Multiplex Fluorescence Melting Curve Analysis for Mutation Detection with Dual-Labeled, Self-Quenched Probes. PLoS One 2011, 6 (4).
(25) Zhan, Y.; Zhang, J.; Yao, S.; Luo, G. High-Throughput Two-Dimensional Polymerase Chain Reaction Technology. Anal. Chem. 2020.
(26) Enroth, C. H.; Fehler, A. O.; Poulsen, L. D.; Vinther, J. Excess Primer Degradation by Exo i Improves the Preparation of 3′ CDNA Ligation-Based Sequencing Libraries. Biotechniques 2019, 67 (3), 110-116.
(27) Corman, V. M.; Landt, O.; Kaiser, M.; Molenkamp, R.; Meijer, A.; Chu, D. K. W.; Bleicker, T.; Brunink, S.; Schneider, J.; Schmidt, M. L.; Mulders, D. G. J. C.; Haagmans, B. L.; Van Der Veer, B.; Van Den Brink, S.; Wijsman, L.; Goderski, G.; Romette, J. L.; Ellis, J.; Zambon, M.; Peiris, M.; Goossens, H.; Reusken, C.; Koopmans, M. P. G.; Drosten, C. Detection of 2019 Novel Coronavirus (2019-NCoV) by Real-Time RT-PCR. Eurosurveillance 2020, 25 (3), 1-8.
(28) Fozouni, P.; Son, S.; Diaz de Le6n Derby, M.; Knott, G. J.; Gray, C. N.; D'Ambrosio, M. V.; Zhao, C.; Switz, N. A.; Kumar, G. R.; Stephens, S. I.; Boehm, D.; Tsou, C. L.; Shu, J.; Bhuiya, A.; Armstrong, M.; Harris, A. R.; Chen, P. Y.; Osterloh, J. M.; Meyer-Franke, A.; Joehnk, B.; Walcott, K.; Sil, A.; Langelier, C.; Pollard, K. S.; Crawford, E. D.; Puschnik, A. S.; Phelps, M.; Kistler, A.; DeRisi, J. L.; Doudna, J. A.; Fletcher, D. A.; Ott, M. Amplification-Free Detection of SARS-CoV-2 with CRISPR-Casl3a and Mobile Phone Microscopy. Cell 2020, 323-333.
(29) Chong, B. S. W.; Tran, T.; Druce, J.; Ballard, S. A.; Simpson, J. A.; Catton, M. Sample Pooling Is a Viable Strategy for SARS-CoV-2 Detection in Low-Prevalence Settings. Pathology 2020, 52 (7), 796-800.
(30) Elnifro, E. M.; Ashshi, A. M.; Cooper, R. J.; Klapper, P. E. Multiplex PCR: Optimization and Application in Diagnostic Virology. Clin. Microbiol. Rev. 2000, 13 (4), 559-570.
(31) Lobato, I. M.; O'Sullivan, C. K. Recombinase Polymerase Amplification: Basics, Applications and Recent Advances. TrAC—Trends Anal. Chem. 2018, 98, 19-35.

We claim:

1. A method of sample pooling of at least two samples, the method comprising:
a) combining a first sample with a first ID-Primer and a second sample with a second ID-Primer wherein:
i) the first ID-Primer comprises a first nucleic acid sequence having sufficient complementarity to a first site in a target nucleic acid operably linked to an amplifier region and a first unique ID region, and
ii) the second ID-Primer comprises a second nucleic acid sequence having sufficient complementarity to the first site in the target nucleic acid operably linked to an amplifier region and a second unique ID region;
b) hybridizing or ligating the first ID primer and the second ID-Primer to the target nucleic acid sequence in the first and second samples or reverse transcribing the target nucleic acid sequence in the first and second samples using a reverse transcriptase and the first ID-Primer or the second ID-Primer; and
c) digesting unused first and second ID-Primers with an exonuclease;
d) pooling the first and second samples together; and
e) optionally, detecting the target nucleic acid sequence.

2. The method of claim 1, wherein the reverse transcriptase is an RNase H Minus reverse transcriptase.

3. The method of claim 1, wherein the exonuclease is exonuclease I.

4. The method of claim 1, wherein target nucleic acid sequence is RNA or DNA.

5. The method of claim 1, wherein the first ID-Primer and the second ID-Primer have a melting temperature that is about 5° C. to about 15° C. or about 10° C. different.

6. The method of claim 1, where the first and second ID-Primers have a different length from each other of least 5, 6, 7, 8, 9, 10, 15, 20 or more nucleotides.

7. The method of claim 1, wherein detecting the target nucleic acid sequence comprises using real time-polymerase chain reaction (RT-PCR).

8. The method of claim 7, wherein detecting the target nucleic acid sequence comprises adding to the pooled sample at least two fluorophore-labelled probes, a first F probe and a second F probe, and at least one quencher-labelled probe (Q probe), wherein the label of the first F probe comprises a fluorescent label and is complementary to the first unique region of the first ID-Primer and the second F probe comprises the fluorescent label and is complementary to the second unique region of the second ID-Primer, and the Q probe comprises a quencher label and is complementary to a region of the target nucleic acid adjacent to the first site of the target nucleic acid.

9. The method of claim 8, wherein detection of the target nucleic acid sequence comprises using RT-PCR comprises:
i) adding a polymerase and a plurality of primers comprising a forward primer and at least two distinct reverse primers to the pooled sample to provide a reaction mixture;
ii) amplifying the target nucleic acid sequence, if present, in the reaction mixture, to generate single-stranded amplicons of the target nucleic acid sequence;
iii) hybridizing the at least two F probes and the Q probe to the single-stranded amplicons, wherein the first and second F probes have a lower melting temperature than the Q probe, and the Q probe has a quencher label that quenches the fluorescence of the respective F probe when the respective F probe and Q probe are hybridized to the single-stranded amplicons of the target nucleic acid;
iv) increasing the temperature of the reaction mixture until the respective F probe is released from the single-stranded amplicon; and
v) detecting the fluorescence of the released F probe.

10. The method of claim 9, wherein the reaction mixture further comprises at least one or more reagents selected from the group consisting of a buffer, a nucleotide, a deoxynucleotide, and a DNA polymerase.

11. The method of claim 8, wherein the fluorescent label of F probe is labelled at the 3′end.

12. The method of claim 8, wherein the quencher of Q probe is labelled at the 5′end.

13. The method of claim 8, wherein the melting temperature of the Q probe is about 70° C. to about 80° C.

14. The method of claim 8, wherein the Q probe has a 3′ inverted dT.

15. The method of claim 9, wherein a first reverse primer is complementary to the amplifier region of the first ID-Primer, and a second reverse primer is complementary to the amplifier region of the second ID-Primer.

16. The method of claim 9, wherein the forward primer is complementary to the target nucleic acid sequence.

17. The method of claim 7, wherein detecting the target nucleic acid sequence comprises adding to the pooled sample at least two fluorophore-labelled and quencher-labelled probes, wherein the label of a first probe comprises a first fluorescent label and is complementary to the first unique region of the first ID-Primer and the label of a second probe comprises a second fluorescent label and is complementary to the second unique region of the second ID-Primer.

18. The method of claim 17, wherein the detecting the target nucleic acid sequence comprises:
i) adding to the pooled samples a polymerase, at the least two fluorophore-labelled and quencher-labelled probes, a plurality of primers comprising a forward primer and at least two distinct reverse primers to provide a reaction mixture, wherein a first reverse primer is complementary to the amplifier region of the first ID-Primer, and a second reverse primer is complementary to the amplifier region of the second ID-Primer;
ii) amplifying the target nucleic acid sequence, if present, in the reaction mixture, whereby the probes are hydrolyzed by exonuclease activity of the polymerase; and
iii) detecting the fluorescence of the released fluorescent label.

19. The method of claim 18, wherein the fluorescent label of the first or second probe is labelled at the 3′end and the quencher of the first or second probe is labelled at the 5′end.

20. The method of claim 1, wherein detecting the target nucleic acid sequence comprises nucleic acid isothermal amplification and detection.

21. The method of claim 20, wherein the nucleic acid isothermal amplification and detection is Loop-mediated isothermal Amplification (LAMP), Recombinase Polymerase Amplification (RPA), Rolling Circle Amplification (RCA), or Nucleic Acid Sequence-based Amplification (NASBA).

22. The method of claim 6, wherein detecting the target nucleic acid sequence comprises a size separation technique.

23. The method of claim 22, wherein the size separation technique comprises adding to the pooled sample at least two reverse primers, a first reverse primer and a second reverse primer, and at least one forward primer, wherein the first reverse primer is complementary to a first amplifier region of the first ID-Primer and the second reverse primer is complementary to a second amplifier region of the second ID-Primer.

24. The method of claim 23, wherein the size separation technique comprises:
i) adding to the pooled samples a DNA polymerase and a plurality of primers comprising the forward primer and the at least two distinct reverse primers to provide a reaction mixture;
ii) amplifying the target nucleic acid sequence, if present, in the reaction mixture, whereby double stranded amplicons with unique lengths are produced; and
iii) detecting the size of the amplicons.

25. The method of claim 24, wherein the size of the amplicon is detected using gel electrophoresis, a fragment analyzer, or a bioanalyzer.

26. The method of claim 1, wherein detecting the target nucleic acid sequence comprises electrochemical detection.

27. The method of claim 26, wherein the electrochemical detection comprises adding to the pooled sample probe with an electroactive reporter and at least two reverse primers, a first reverse primer and a second reverse primer, and at least one forward primer, wherein the first reverse primer is complementary to a first amplifier region of the first ID-Primer and the second reverse primer is complementary to a second amplifier region of the second ID-Primer.

28. The method of claim 27, wherein the electrochemical detection comprises:
i) adding to the pooled samples a polymerase, a probe with an electroactive reporter, and a plurality of primers comprising the forward primer and the at least two distinct reverse primers to provide a reaction mixture;
ii) amplifying the target nucleic acid sequence, if present, in the reaction mixture, whereby the electroactive reporter is released via exonuclease activity of the polymerase; and
iii) detecting an electroactive signal of the released electroactive reporter.

29. The method of claim 27, wherein electrochemical detection is an end point test.

30. The method of claim 28, wherein, the electrochemical signal is generated from methylene blue, ferrocene, or another electroactive reporter.

31. A method of sample pooling of at least two samples, the method comprising:
a) combining a first sample with a first ID-Primer and at least one labeled capture strand and a second sample with a second ID-Primer and at least one labeled capture strand wherein:
i) the first ID-Primer comprises a first nucleic acid sequence having sufficient complementarity to a first site in a target nucleic acid operably linked to an amplifier region and a first unique ID region,
ii) the second ID-Primer comprises a second nucleic acid sequence having sufficient complementarity to the first site in the target nucleic acid operably linked to an amplifier region and a second unique ID region, wherein the first ID-Primer and the second ID-Primers have distinguishable melting temperatures, and
iii) the label of the capture strand comprises a biotin label and is complementary to the target nucleotide sequence adjacent to the first site or the second site in the target nucleic acid;
b) attaching the biotin labeled capture strand to a streptavidin coated magnetic bead;
c) hybridizing the first ID primer, the second ID-Primer, and the at least one capture strand to the target nucleic acid sequence in the first and second samples;
d) washing away unbound primers and capture strands;
e) pooling the first and second sample together; and
f) optionally, detecting the target nucleic acid sequence.

32. The method of claim 31, wherein detecting the target nucleic acid sequence comprises:
i) adding to the pooled samples to a reverse transcriptase;
ii) reverse transcribing the target nucleic acid sequence, if present, in the reaction mixture, whereby the bound capture strand is displaced; and
iii) separating unbound capture strand from the reverse transcribed target nucleic acid using a magnetic field.

33. The method of claim 31, wherein detecting the target nucleic acid sequence comprises real time PCR (RT-PCR), nucleic acid isothermal amplification and detection, size separation technique, or electrochemical detection.


SEQUENCE LISTING (22 sequences)

SEQ ID NO: 1 - RNA, 880 nt, Artificial Sequence
  Synthetic RNA(EURO-019) sequence
gggagacgaauugggcccucuagaugcaugcucgagcggccgccagugugauggauaucugcagaauucgcccuuauucaaguauugagugaaauggucauguguggcgguucacuauauguuaaaccagguggaaccucaucaggagaugccacaacugcuuaugcuaauaguguuuuuaacauuugucaagcuguccggaagagacagguacguuaauaguuaauagcguacuucuuuuucuugcuuucgugguauucuugcuaguuacacuagccauccuuacugcgcuucgauugugugcguacugcugcaauauuguuaacguauaauggaccccaaaaucagcgaaaugcaccccgcauuacguuugguggacccucagauucaacuggcaguaaccagaauggagaacgcauugcaacugagggagccuugaauacaccaaaagaucacauuggcacccgcaauccugcuaacaaugcugcaaucgugcuacaacuuccucaaggaaauuuuggggaccaggaacuaaucagacaaggaacugauuacaaacauuggccgcaaauugcacaauuugcccccagcgcuucagcguucuucggaaugucgcgcauuggcauggaagucacaccuucgggaacgugguugaccuacacaggugccaucaaauuggagugugacauacccauuggugcagguauaugcgcuaguuaucagacucagacuaauucuccucggcgggcacguaguguagcuagucaaccugcuuuugcucgcuuggauccgaauucaaaggugaaauuguuauccgcucacaauuccacacaacauacgagccggaagcauaaaguguaaagccuggggugccuaauga

SEQ ID NO: 2 - DNA, 25 bp, Artificial Sequence
  PCR universal Forward primer to amplify templates with ID-Primers
cgtataatggaccccaaaatcagcg

SEQ ID NO: 3 - DNA, 61 bp, Artificial Sequence
  Multiplex ID-Primer 1 for the first sample in one pool
gcagagtcggcctacaggtttatgtatcataagtcaaatgtctggttactgccagttgaat

SEQ ID NO: 4 - DNA, 71 bp, Artificial Sequence
  Multiplex ID-Primer 2 for the second sample in one pool
ctcactgcctactactccacatagcaactgtaagttttatggccttctcttctggttactgccagttgaat

SEQ ID NO: 5 - DNA, 72 bp, Artificial Sequence
  Multiplex ID-Primer 3 for the third sample in one pool
ctctcaacctccaccccttcagtctgctaaggtcatacgtccccttggctctctggttactgccagttgaat

SEQ ID NO: 6 - DNA, 67 bp, Artificial Sequence
  Multiplex ID-Primer 4 for the fourth sample in one pool
ccattagaacctaagctactccacgctatgtatcataagtcaaatgtctggttactgccagttgaat

SEQ ID NO: 7 - DNA, 69 bp, Artificial Sequence
  Multiplex ID-Primer 5 for the fifth sample in one pool
cgagagccaggtaacgaatggtcatgttgttaaggagcgaattaaatctctggttactgccagttgaat

SEQ ID NO: 8 - DNA, 21 bp, Artificial Sequence
  Reverse Primer 1 to amplify samples with ID-Primer 1
gcagagtcggcctacaggttt

SEQ ID NO: 9 - DNA, 26 bp, Artificial Sequence
  Reverse Primer 2 to amplify samples with ID-Primer 2
ctcactgcctactactccacatagca

SEQ ID NO: 10 - DNA, 21 bp, Artificial Sequence
  Reverse Primer 3 to amplify samples with ID-Primer 3
ctctcaacctccaccccttca

SEQ ID NO: 11 - DNA, 27 bp, Artificial Sequence
  Reverse Primer 4 to amplify samples with ID-Primer 4
ccattagaacctaagctactccacgct

SEQ ID NO: 12 - DNA, 22 bp, Artificial Sequence
  Reverse Primer 5 to amplify samples with ID-Primer 5
cgagagccaggtaacgaatggt

SEQ ID NO: 13 - DNA, 19 bp, Artificial Sequence
  Fluorophore probe 1 to detect amplicons with ID-Primer 1
  FAM label (19)..(19)
atgtatcataagtcaaatg

SEQ ID NO: 14 - DNA, 24 bp, Artificial Sequence
  Fluorophore probe 2 to detect amplicons with ID-Primer 2
  FAM label (24)..(24)
actgtaagttttatggccttctct

SEQ ID NO: 15 - DNA, 30 bp, Artificial Sequence
  Fluorophore probe 3 to detect amplicons with ID-Primer 3
  FAM label (30)..(30)
gtctgctaaggtcatacgtccccttggctc

SEQ ID NO: 16 - DNA, 19 bp, Artificial Sequence
  Fluorophore probe 4 to detect amplicons with ID-Primer 4
  HEX label (19)..(19)
atgtatcataagtcaaatg

SEQ ID NO: 17 - DNA, 26 bp, Artificial Sequence
  Fluorophore probe 5 to detect amplicons with ID-Primer 5
  HEX label (26)..(26)
catgttgttaaggagcgaattaaatc

SEQ ID NO: 18 - DNA, 40 bp, Artificial Sequence
  Quencher probe to detect amplicons
  BHQ2 label (1)..(1)
ctgagggtccaccaaacgtaatgcggggtgcatttcgctg

SEQ ID NO: 19 - RNA, 1261 nt, Human coronavirus
augucugauaauggaccccaaaaucagcgaaaugcaccccgcauuacguuugguggacccucagauucaacuggcaguaaccagaauggagaacgcaguggggcgcgaucaaaacaacgucggccccaagguuuacccaauaauacugcgucuugguucaccgcucucacucaacauggcaaggaagaccuuaaauucccucgaggacaaggcguuccaauuaacaccaauagcaguccagaugaccaaauuggcuacuaccgaagagcuaccagacgaauucgugguggugacgguaaaaugaaagaucucaguccaagaugguauuucuacuaccuaggaacugggccagaagcuggacuucccuauggugcuaacaaagacggcaucauauggguugcaacugaugggagccuugaauacaccaaaagaucacauuggcacccgcaauccugcuaacaaugcugcaaucgugcuacaacuuccucaaggaacaacauugccaaaaggcuucuacgcagaagggagcagaggcggcagucaagccucuucucguuccucaucacguagucgcaacaguucaagaaauucaacuccaggcagcaguaggggaacuucuccugcuagaauggcuggcaauggcggugaugcugcucuugcuuugcugcugcuugacagauugaaccagcuugagagcaaaaugucugguaaaggccaacaacaacaaggccaaacugucacuaagaaaucugcugcugaggcuucuaagaagccucggcaaaaacguacugccacuaaagcauacaauguaacacaagcuuucggcagacgugguccagaacaaacccaaggaaauuuuggggaccaggaacuaaucagacaaggaacugauuacaaacauuggccgcaaauugcacaauuugcccccagcgcuucagcguucuucggaaugucgcgcauuggcauggaagucacaccuucgggaacgugguugaccuacacaggugccaucaaauuggaugacaaagauccaaauuucaaagaucaagucauuuugcugaauaagcauauugacgcauacaaaacauucccaccaacagagccuaaaaaggacaaaaagaagaaggcugaugaaacucaagccuuaccgcagagacagaagaaacagcaaacugugacucuucuuccugcugcagauuuggaugauuucuccaaacaauugcaacaauccaugagcagugcugacucaacucaggccuaa

SEQ ID NO: 20 - DNA, 20 bp, Artificial Sequence
  N gene forward primer to amplify SARS-CoV-2 N Gene
gaccccaaaatcagcgaaat

SEQ ID NO: 21 - DNA, 24 bp, Artificial Sequence
  N gene reverse primer to amplify SARS-CoV-2 N Gene
tctggttactgccagttgaatctg

SEQ ID NO: 22 - DNA, 24 bp, Artificial Sequence
  N gene TaqMan probe to detect SARS-CoV-2 N Gene
  FAM label (1)..(1)
  BHQ2 label (24)..(24)
accccgcattacgtttggtggacc